(19) United States
(12) Patent Application Publication
(10) Pub. No.: US 2025/0312349 A1
(43) Pub. Date: Oct. 9, 2025

(54) COMPOSITION AND METHODS OF TREATING INTRACELLULAR PATHOGEN INFECTION

(71) Applicant: UNIVERSITY HOSPITALS CLEVELAND MEDICAL CENTER, Cleveland, OH (US)

(72) Inventor: Carlos Subauste, Shaker Heights, OH (US)

(21) Appl. No.: 19/171,916

(22) Filed: Apr. 7, 2025

Related U.S. Application Data
(60) Provisional application No. 63/575,169, filed on Apr. 5, 2024.

(51) Int. Cl. A61K 31/517; A61K 31/42; A61K 31/505; A61K 31/5377; A61P 33/02

(52) U.S. Cl. CPC A61K 31/517; A61K 31/42; A61K 31/505; A61K 31/5377; A61P 33/02

(57) ABSTRACT

A method of inducing killing and/or degradation of an intracellular pathogen in a subject in need thereof includes administering to the subject an amount of an Src family kinase inhibitor effective to inhibit pathogen activation of Src and AKT in the subject.

### RELATED APPLICATION

This application claims priority from U.S. Provisional Application No. 63/575,169, filed Apr. 5, 2024, the subject matter of which is incorporated herein by reference in its entirety.

### SEQUENCE LISTING

The instant application contains a Sequence Listing which has been submitted electronically in XML format and is hereby incorporated by reference in its entirety. Said XML copy, created on Apr. 4, 2025, is named UH-033452US ORD.st.26 and is 4,653 bytes in size.

### BACKGROUND

Intracellular pathogens utilize a variety of strategies to manipulate host cells to establish a niche that ensures their survival and proliferation. The obligate intracellular protozoan, Toxoplasma gondii, is an excellent example of a pathogen that survives within the host by deploying strategies that include avoidance of host cell-autonomous mechanisms of defense, manipulation of signal transduction in the host to affect the immune response, and blocking apoptosis of infected cells. Indeed, this highly successful pathogen causes chronic infection in approximately 30% of the world population. T. gondii is important clinically because it is a major cause of infectious retinitis worldwide, can cause encephalitis in immunosuppressed individuals, and lead to congenital toxoplasmosis. While antibiotics are available against toxoplasmosis, there is no evidence that they positively affect visual outcome or disease recurrence in the case of ocular toxoplasmosis. In addition, antimicrobial agents used for the treatment of cerebral or ocular toxoplasmosis can result in significant side effects.

T. gondii resides within host cells in a parasitophorous vacuole (PV) that must avoid lysosomal degradation mediated by macroautophagy. Macroautophagy, herein referred to as autophagy, is a process in which cellular cargo is directed to the lysosomal compartment for degradation. This process initiates with the formation of the phagophore, a precursor of the double-membrane autophagosome, which is necessary for the sequestration of cargo, such as intracellular pathogens. The phagophore elongates around the sequestered cargo as a result of the assembly of core autophagy machinery proteins that function as a ubiquitin-like conjugation system. This results in the formation of a double membrane autophagosome that encircles the cargo, which is followed by fusion with lysosomes and cargo degradation.

Autophagy is a constitutive process, indicating that T. gondii must employ strategies to avoid targeting by autophagosomes. Indeed, the parasite activates EGFR within the infected host cell to avoid targeting by autophagy. Inhibition of EGFR signaling in EGFR+ cells either by expression of dominant negative (DN) EGFR or by treatment with EGFR tyrosine kinase inhibitors (TKI) induces autophagic killing of T. gondii. Moreover, treatment of mice with ocular and cerebral toxoplasmosis with Gefitinib, an EGFR TKI, results in partial reduction in parasite load and histopathology in the eye and brain that are dependent on Beclin 1. Despite in vitro and in vivo studies supporting the relevance of EGFR activation as a strategy for T. gondii survival, it is important to emphasize that the expression of EGFR is not widespread. In this regard, expression of EGFR in normal adult neural tissue (the main site affected in toxoplasmosis) is moderate and restricted to areas such as the EGF subventricular zone. This is likely to explain why EGFR inhibition only causes a moderate reduction in parasite load and histopathology in the eye and brain, and would point towards the existence of a mechanism to avoid autophagic targeting that allows parasite survival in cells that lack EGFR.

### SUMMARY

Embodiments described herein relate to compositions and methods of inducing killing and/or degradation of an intracellular pathogen, such as Toxoplasma gondii, in a subject in need thereof, and particularly relate to compositions and methods of treating toxoplasmosis, such as ocular and/or neural toxoplasmosis, as well retinitis and encephalitis caused by intracellular pathogen infection, e.g., toxoplasmosis, in a subject in need thereof.

T. gondii, a causative agent of retinitis and encephalitis, survives within host cells by avoiding autophagy-dependent degradation by the lysosome. While T. gondii activates EGFR to avoid autophagic killing, EGFR expression is limited in neural tissue. We found that, independently of EGFR, T. gondii activates the ubiquitous molecule, Src, resulting in PTEN inhibition and decoration of the parasite-containing vacuole with activated Akt (negative regulator of autophagy). Inhibition of Src, by Src knockdown or treatment with an Src family kinase inhibitor, impaired this cascade, leading to autophagic killing of T. gondii demonstrated by autophagosomal and lysosomal marker recruitment and parasite killing dependent on ULK1 and lysosomal enzymes. Treatment with the Src family kinase inhibitor induced PTEN recruitment around parasites in neural tissue and impaired recruitment of activated Akt, causing a striking reduction in parasite load and histopathology in mice with ocular and cerebral toxoplasmosis. Autophagy-deficient mice treated with an Src family kinase inhibitor did not have improved parasite load or histopathology, supporting the pivotal role of this pathway for parasite survival and development of toxoplasmosis.

In some embodiments, a method of inducing killing and/or degradation of an intracellular pathogen that activates Src and AKT to avoid killing and/or degradation of the intracellular pathogen in a subject in need thereof can include administering to the subject an amount of an Src family kinase inhibitor effective to inhibit pathogen activation of Src and AKT in the subject.

In some embodiments, the intracellular pathogen can include a parasitic protozoan, such as an apicomplexan.

In some embodiments, the apicomplexan can include T. gondii.

In some embodiments, the subject has toxoplasmosis, such as neural toxoplasmosis, for example, ocular and/or cerebral toxoplasmosis.

In other embodiments, the subject has retinitis and/or encephalitis caused by ocular and/or cerebral toxoplasmosis.

In some embodiments, the Src family kinase inhibitor is administered at a therapeutic dose or subtherapeutic dose.

In some embodiments, the Src family kinase inhibitor comprises at least one of N-(2-chloro-6-methylphenyl)-2-[[6-[4-(2-hydroxyethyl)-1-piperazinyl]-2-methyl-4-pyrimidinyl]amino]-5-thiazole carboxamide monohydrate (dasatinib), N-(5-chloro-1,3-benzodioxol-4-yl)-7-(2-(4-methylpiperazin-1-yl)ethoxy)-5-(tetrahydro-2H-pyran-4-yloxy)quinazolin-4-amine (saracatinib), 4-[(2,4-dichloro-5-methoxyphenyl)amino]-6-methoxy-7-[3-(4-methylpiperazin-1-yl)propoxy]quinoline-3-carbonitrile (bosutinib), (4-Amino-5-(4-methylphenyl)-7-(t-butyl)pyrazolo[3,4-d]-pyrimidine), (4-chlorophenyl)-7-(t-butyl)pyrazolo[3,4-d]-pyrimidine) (PP1), 1-tert-butyl-3-(4-chlorophenyl)pyrazolo[3,4-d]pyrimidin-4-amine (PP2), 6-(2,6-dichlorophenyl)-2-{[3-(hydroxymethyl)phenyl]amino}-8-methyl-7H,8H-pyrido[2,3-d]pyrimidin-7-one (PD1663266), (E)-N-[4-[3-chloro-4-(pyridin-2-ylmethoxy)anilino]-3-cyano-7-ethoxyquinolin-6-yl]-4-(dimethylamino)but-2-enamide (neratinib), 3-(2-imidazo[1,2-b]pyridazin-3-ylethynyl)-4-methyl-N-[4-[(4-methylpiperazin-1-yl)methyl]-3-(trifluoromethyl)phenyl]benzamide (ponatinib), (E)-N-[4-(3-chloro-4-fluoroanilino)-3-cyano-7-ethoxyquinolin-6-yl]-4-(dimethylamino)but-2-enamide (pelitinib), N-benzyl-2-[5-[4-(2-morpholin-4-ylethoxy)phenyl]pyridin-2-yl]acetamide (Tirbanibulin), 4-methyl-3-[(2-methyl-6-pyridin-3-ylpyrazolo[3,4-d]pyrimidin-4-yl)amino]-N-[3-(trifluoromethyl)phenyl]benzamide (NVP-BHG712), (2S,3S)-2,3-dihydroxybutanedioic acid; 6-(4-methylpiperazin-1-yl)-N-(5-methyl-1H-pyrazol-3-yl)-2-[(E)-2-phenylethenyl]pyrimidin-4-amine (ENMD-2076), 4-[4-[(5-tert-butyl-2-quinolin-6-ylpyrazol-3-yl)carbamoylamino]-3-fluorophenoxy]-N-methylpyridine-2-carboxamide (Rebastinib), analogues thereof, or any combination thereof. Preferably, the Src family kinase inhibitor is saracatinib (AZD0530) or an analogue thereof.

In some embodiments, the Src family kinase inhibitor can be administered to the subject at a dose of less than about 150 mg per day, less than about 140 mg per day, less than about 130 mg per day, less than about 120 mg per day, less than about 110 mg per day, less than about 100 mg per day, less than about 90 mg per day, less than about 80 mg per day less than about 70 mg per day, less than about 60 mg per day, or less than about 50 mg per day.

In some embodiments, the Src family kinase inhibitor can be administered at an amount effective to provide a plasma drug level less than about 250 ng/ml, less than about 200 ng/ml, less than about 150 ng/ml, or less than about 100 ng/ml.

In some embodiments, the method further includes administering an antimicrobial agent and/or EGFR inhibitor in combination with the Src family kinase inhibitor.

In some embodiments, the antimicrobial agent can include an antiprotozoal agent.

In some embodiments, the EGFR inhibitor is an EGFR tyrosine kinase inhibitor.

In some embodiments, the antimicrobial agent and/or EGFR inhibitor is administered at a subtherapeutic dose with a subtherapeutic dose of the Src family kinase inhibitor. The killing and/or degradation effect of the Src inhibitor on the pathogen is enhanced as compared to the effect of the Src inhibitor administered without the antimicrobial agent and/or EGFR inhibitor.

In some embodiments, the antimicrobial agent can include at least one of amoxicillin, atovaquone, diaminopyrimidines, especially amodiaquine, amphotericin, proguanil (chlorguanide), chloroquine, clindamycin, eflornithine, furazolidone, a fluoroquinolone, such as ciprofloxacin or levofloxacin, or a third generation cephalosporin, such as ceftriaxone or cefixime, hydroxychloroquine, mefloquine, melarsoprol, metronidazole, minocycline, nifursemizone, nitazoxanide, ornidazole, paromycin sulfate, pentamidine, pyrimethamine, quinapyramine, ronidazole, tinidazole, spriramycin, sulfadiazine, sulfamethoxazole, trimethoprim, analogues thereof, or combinations thereof.

In some embodiments, the EGFR inhibitor can include at least one of gefitinib (Iressa), erlotinib (Tarceva), afatinib (Gilotrif), dacomitinib (Vizimol), or osimertinib (Targrisso).

Other embodiments described herein relate to an Src family kinase inhibitor in combination with an antimicrobial agent and/or EGFR inhibitor for use in treating an intracellular pathogen infection. The Src family kinase inhibitor, the antimicrobial agent, and/or the EGFR inhibitor can be formulated and administered at an amount effective to induce killing and/or degradation of the pathogen in a subject in need thereof.

In some embodiments, the antimicrobial agent and/or EGFR inhibitor can be formulated and administered at a subtherapeutic dose with a subtherapeutic dose of the Src family kinase inhibitor. The killing and/or degradation effect of the Src family kinase inhibitor on the pathogen can be enhanced as compared to the effect of the Src family kinase inhibitor administered without the antimicrobial agent and/or EGFR inhibitor.

In some embodiments, the intracellular pathogen treated by the combination activates Src and AKT to avoid killing in the subject.

In some embodiments, the intracellular pathogen can include a parasitic protozoan, such as an apicomplexan.

In some embodiments, the intracellular pathogen includes Toxoplasma gondii.

In some embodiments, the subject has toxoplasmosis, such as neural toxoplasmosis, for example, ocular and/or cerebral toxoplasmosis.

In other embodiments, the subject has retinitis and/or encephalitis caused by ocular and/or cerebral toxoplasmosis.

In some embodiments, the Src family kinase inhibitor comprises at least one of N-(2-chloro-6-methylphenyl)-2-[[6-[4-(2-hydroxyethyl)-1-piperazinyl]-2-methyl-4-pyrimidinyl]amino]-5-thiazole carboxamide monohydrate (Dasatinib), N-(5-chloro-1,3-benzodioxol-4-yl)-7-(2-(4-methylpiperazin-1-yl)ethoxy)-5-(tetrahydro-2H-pyran-4-yloxy)quinazolin-4-amine (saracatinib), 4-[(2,4-dichloro-5-methoxyphenyl)amino]-6-methoxy-7-[3-(4-methylpiperazin-1-yl)propoxy]quinoline-3-carbonitrile (bosutinib), (4-Amino-5-(4-methylphenyl)-7-(t-butyl)pyrazolo[3,4-d]-pyrimidine), (4-chlorophenyl)-7-(t-butyl)pyrazolo[3,4-d]-pyrimidine) (PP1), 1-tert-butyl-3-(4-chlorophenyl)pyrazolo[3,4-d]pyrimidin-4-amine (PP2), 6-(2,6-dichlorophenyl)-2-{[3-(hydroxymethyl)phenyl]amino}-8-methyl-7H,8H-pyrido[2,3-d]pyrimidin-7-one (PD1663266), (E)-N-[4-[3-chloro-4-(pyridin-2-ylmethoxy)anilino]-3-cyano-7-ethoxyquinolin-6-yl]-4-(dimethylamino)but-2-enamide (neratinib), 3-(2-imidazo[1,2-b]pyridazin-3-ylethynyl)-4-methyl-N-[4-[(4-methylpiperazin-1-yl)methyl]-3-(trifluoromethyl)phenyl]benzamide (ponatinib), (E)-N-[4-(3-chloro-4-fluoroanilino)-3-cyano-7-ethoxyquinolin-6-yl]-4-(dimethylamino)but-2-enamide (pelitinib), N-benzyl-2-[5-[4-(2-morpholin-4-ylethoxy)phenyl]pyridin-2-yl]acetamide (Tirbanibulin), 4-methyl-3-[(2-methyl-6-pyridin-3-ylpyrazolo[3,4-d]pyrimidin-4-yl)amino]-N-[3-(trifluoromethyl)phenyl]benzamide (NVP-BHG712), (2S,3S)-2,3-dihydroxybutanedioic acid; 6-(4-methylpiperazin-1-yl)-N-(5-methyl-1H-pyrazol-3-yl)-2-[(E)-2-phenylethenyl]pyrimidin-4-amine (ENMD-2076), 4-[4-[(5-tert-butyl-2-quinolin-6-ylpyrazol-3-yl)carbamoylamino]-3-fluorophenoxy]-N-methylpyridine-2-carboxamide (Rebastinib), analogues thereof, or any combination thereof. Preferably, the Src family kinase inhibitor is saracatinib (AZD0530) or an analogue thereof.

In some embodiments, the antimicrobial agent can include at least one of amoxicillin, atovaquone, diaminopyrimidines, especially amodiaquine, amphotericin, proguanil (chlorguanide), chloroquine, clindamycin, eflornithine, furazolidone, a fluoroquinolone, such as ciprofloxacin or levofloxacin, or a third generation cephalosporin, such as ceftriaxone or cefixime, hydroxychloroquine, mefloquine, melarsoprol, metronidazole, minocycline, nifursemizone, nitazoxanide, ornidazole, paromycin sulfate, pentamidine, pyrimethamine, quinapyramine, ronidazole, tinidazole, spriramycin, sulfadiazine, sulfamethoxazole, trimethoprim, analogues thereof, or combinations thereof.

In some embodiments, the EGFR inhibitor can include cetuximab, panitumumab, erlotinib, gefitinib, tyrphostins, or combinations thereof.

In other embodiments, the EGFR inhibitor can include at least one of gefitinib (Iressa), erlotinib (Tarceva), afatinib (Gilotrif), dacomitinib (Vizimol), or osimertinib (Targrisso).

### BRIEF DESCRIPTION OF THE DRAWINGS

FIGS. 1(A-D) illustrate graphs, plots, and assays showing the inhibition of Src triggers killing of T. gondii in cells that lack functional EGFR. (A) EGFR mRNA levels in parental CHO cells and CHO cells stably transfected to express EGFR were measured by RT-qPCR using 18S rRNA as an internal control. One sample from parental cells was given an arbitrary value of 1, and data are expressed as fold-increase compared to this sample. Graph represents mean±SEM from 3 independent samples per cell type. Cell lysates were probed for EGFR and actin. Relative density of EGFR was obtained by normalization relative to the parental CHO sample. Densitometry graph represents mean±SEM from 3 independent experiments. (B) CHO cells and primary mouse brain endothelial cells that express dominant negative (DN) EGFR were challenged with RH T. gondii. CHO cells were lysed at the indicated times, whereas brain endothelial cells were lysed after 2 h. Lysates were probed for Src and phospho-Y416 Src. Relative density of phospho-Src was obtained by normalization to total Src followed by normalization relative to the uninfected sample. Relative density of phospho-Src for uninfected sample was given a value of 1. Data are shown as mean±SEM from 2 or 3 independent experiments. (C, D) CHO cells (C), and primary mouse brain endothelial cells expressing WT EGFR or DN EGFR (D) transfected with Ctr or Src siRNA were challenged with RH T. gondii and examined at 2 and 24 h. Results are shown as mean±SEM. The graphs show data from 6 independent monolayers pooled from 3 different experiments. Data points are shown as circles, triangles, or squares corresponding to individual monolayers within each experiment. All significance was determined using two-tailed, unpaired Student's t test comparing six independent monolayers per group (**p<0.01,***p<0.001, ****p<0.0001).

FIGS. 2(A-G) illustrate graphs, plots, and assays showing the inhibition of T. gondii-induced Src signaling triggers parasite killing even in cells that lack functional EGFR. (A) CHO cells were challenged with RH T. gondii and treated with or without Saracatinib (1 M). Lysates collected at 2 h post-infection were probed for Src and phospho-Y416 Src. Relative density of phospho-Src was normalized to total Src followed by normalization relative to the uninfected sample. Densitometry graph shows mean±SEM from 3 independent experiments. (B) EGFR⁺ CHO cells infected with RH T. gondii were treated with the indicated concentrations of Saracatinib or Gefitinib. Percentages of infection and tachyzoites/100 cells were assessed after 24 h. (C, D) CHO cells were infected with RH (Type 1) or PTG (Type 2) strains of T. gondii. Cells were assessed after 2 h and 24 h. (E) Primary mouse brain endothelial cells expressing WT or DN EGFR were infected with RH T. gondii and treated with or without Saracatinib (1 μM). Cells were evaluated at 24 h. (F) RPE cells were challenged with RH T. gondii and treated with or without Saracatinib (1 μM). Percentage of infected cells and number of tachyzoites per 100 cells were determined by light microscopy 24-hr post-infection. (G) Mouse endothelial cells (mHEVc) transduced with control shRNA or Src shRNA were incubated with or without Saracatinib (1 μM). Data are shown as mean SEM. The graphs show data from 6 independent monolayers pooled from 3 different experiments. Data points are shown as circles, triangles, or squares corresponding to individual monolayers within each experiment. All significance was determined using two-tailed, unpaired Student's t test comparing six independent monolayers per group (***p<0.001, ****p<0.0001).

FIGS. 3(A-L) illustrate images and graphs showing T. gondii killing induced by Src inhibition is mediated by autophagy. (A-C) CHO cells and primary brain endothelial cells expressing WT or DN EGFR were infected with RFP-T. gondii and treated with Saracatinib 1 μM for 5 hours before staining with an anti-LC3 antibody. Accumulation of LC3 around the parasites was assessed by confocal microscopy. Scale bar, 10 μm. (D-F) CHO cells and primary brain endothelial cells expressing WT or DN EGFR were infected with RFP-T. gondii and treated with Saracatinib 1 μM for 7 hours before staining with anti-LAMP1 antibody. Accumulation of LAMP1 around parasites was assessed as above. G-I) CHO cells and primary brain endothelial cells expressing WT or DN EGFR were transfected with control siRNA or ULK1 siRNA. Relative density of ULK1 was normalized to total Actin followed by normalization relative to Ctr siRNA. Densitometry graph shows mean±SEM from 2 independent experiments. Cells were treated with or without Saracatinib and infected with RH T. gondii. Monolayers were examined at 24 h. (J-L) CHO cells and primary brain endothelial cells expressing WT or DN EGFR were infected with RH T. gondii and incubated with or without Saracatinib. Leupeptin plus pepstatin were added post-infection. Data are shown as mean±SEM. The graphs show data from 6 independent monolayers pooled from 3 different experiments. Data points are shown as circles, triangles, or squares corresponding to individual monolayers within each experiment. All significance was determined using two-way unpaired Student's t test comparing 6 individual monolayers per group (**p<0.01,***p<0.001,****p<0.0001).

FIGS. 4(A-C) illustrate Src inhibition induces T. gondii killing by impairing Akt activation. (A) CHO cells were challenged with RH-T. gondii. Cell lysates collected at 2 h were probed for Akt and phospho-S473 Akt. Relative density of phospho-Akt was normalized to total Akt and compared to the uninfected sample. Densitometry graph shown as mean±SEM from 3 independent experiments. (B-C) CHO cells or primary brain endothelial cells were transfected with plasmids encoding HA-tagged CA- or HA-tagged WT-Akt. Lysates were probed for actin, HA, Akt and phospho-S473 Akt. Cells were challenged with T. gondii and treated with Saracatinib (1 μM). Cell lysates collected at 2 h were probed for Akt and phospho-S473 Akt. Relative density of phospho-Akt and densitometries were examined as above. Percentages of infected cells and tachyzoites per 100 cells were assessed at 24 h. Data are shown as mean±SEM. The graphs show data from 6 independent monolayers pooled from 3 different experiments. Data points are shown as circles, triangles, or squares corresponding to individual monolayers within each experiment. Significance was determined using two-way unpaired Student's t test comparing 6 individual monolayers per group (**p<0.01, ***p<0.001, ****p<0.0001).

FIGS. 5(A-F) illustrate graphs, images, and assays showing Src impairs the function of PTEN to regulate Akt signaling. (A) CHO cells were transfected with control or Src siRNA or treated with or without Saracatinib followed by infection with RH T. gondii. Cell lysates were obtained at 2 h and were probed for PI3K and phospho-Y199 PI3K. Relative density of phospho-PI3K was normalized to total PI3K and compared to the uninfected sample. Densitometry analysis of blots shown from 3 independent experiments. (B) CHO cells infected with RFP-T. gondii (magenta) and treated with or without Saracatinib. Cells were stained with antibodies against phospho-S73 Akt (green) and GRA7 (red). DAPI (blue) stains the nucleus. Accumulation of phospho-S73 Akt around the parasites was assessed by confocal microscopy. Scale bar, 10 m. Data are shown as mean±SEM. The graphs show data from 5 independent monolayers pooled from 3 different experiments. Data points are shown as circles, triangles, or squares corresponding to individual monolayers within each experiment. (C) CHO cells were transfected with or without Ctr or Src siRNA or incubated with or without Saracatinib. Cell lysate obtained at 2 h were probed with total PTEN, phospho-S380/T382/383 PTEN or phospho-Y240 PTEN. Densitometry analysis shows means±SEM from 2-3 independent experiments. (D) Infected and uninfected CHO cells were subjected to immunoprecipitation using an anti-Src antibody. Whole cell lysates and immunoprecipitated were probed using antibodies against PTEN and Src. Relative density of PTEN in the immunoprecipitate (IP) was obtained by normalization to total Src in the IP followed by normalization relative to the IP from uninfected sample. Relative density of PTEN for uninfected sample was given a value of 1. Densitometry was assessed as above. (E) CHO cells infected with RFP-T. gondii were stained with antibodies against PTEN and GRA7. DAPI stains the nucleus. Accumulation of PTEN around the parasites was assessed by confocal microscopy. The graphs show data from 6 independent monolayers pooled from 3 different experiments. Data points are shown as circles, triangles, or squares corresponding to individual monolayers within each experiment. (F) CHO cells were transfected with control siRNA or PTEN siRNA prior to challenge with RH T. gondii and incubated with or without Saracatinib. Monolayers were examined at 24 h. Data are shown as mean±SEM. The graphs show data from 6 independent monolayers pooled from 3 different experiments. Data points are shown as circles, triangles, or squares corresponding to individual monolayers within each experiment. Significance was determined using two-way unpaired Student's t test (***p<0.001, ****p<0.0001).

FIGS. 6(A-K) illustrate images and graphs showing Saracatinib confers protection against ocular and cerebral toxoplasmosis. B6 mice were infected with 10 ME49 T. gondii tissue cysts for four weeks before treatment with Saracatinib (10 or 15 mg kg⁻¹ oral gavage twice per day; 5 days per week), TMP-SMX (120:600 mg kg⁻¹ oral gavage once a day; 5 days per week) or vehicle. Mice were euthanized after 4 weeks of treatment (8 weeks total infection). (A-B) Representative images of retinas and brains of chronically infected mice after four weeks of infection prior to Saracatinib, TMP-SMX, or vehicle administration. Retina: arrowhead=vitreal inflammation; arrow=perivascular inflammation; asterix=disruption of retinal architecture. Brain: arrowhead=perivascular inflammation; arrow, tissue cyst; asterix; microglial nodule. Scale bar, 50 m. Data represented as mean±SEM from 8 mice per group pooled from 2-3 independent experiments. (C-D) Representative images of retinas and brains as well as histopathological scoring at 8 weeks post-infection (4 weeks of infection followed by 4 weeks of treatment). Scale bar, 50 m. Data are shown as mean±SEM from mice pooled from 2-3 independent experiments: Retina-n=17 (Ctr), 5 (TMP-SMX), 15 (Saracatinib 10 mg/kg), and 8 (Saracatinib 15 mg/kg). Brain n=15 (Ctr), 5 (TMP-SMX), 17 (Saracatinib 10 mg/kg), and 8 (Saracatinib 15 mg/kg). Statistical significance was determined using two-way ANOVA with Tukey multiple comparison test. (E) T. gondii B1 gene was examined in the eye qPCR. Levels were compared to those of one control mouse that was given an arbitrary value of 1. Bars represent mean±SEM pooled from mice pooled from 2-3 independent experiments: n=11 (Ctr), 4 (TMP-SMX), 10 (Saracatinib 10 mg/kg), and 10 (Saracatinib 15 mg/kg). Statistical significance was determined using one-way ANOVA with Tukey multiple comparison test. (F) Number of T. gondii tissue cysts were counted per brain homogenates. Data are shown as mean±SEM from mice pooled from 2-3 independent experiments: n=11 (Ctr), 5 (TMP-SMX), 9 (Saracatinib 10 mg/kg), and 8 (Saracatinib 15 mg/kg). Statistical significance was determined using one-way ANOVA with Tukey multiple comparison test. (G). Levels of IL-12 p40, IFN-γ, TNF-α and/or NOS2 mRNA in the eye and brain were measured using RT-qPCR. Data shown as mean±SEM from mice pooled from 2 independent experiments: Retina n=6 Ctr, 7 Saracatinib (IFN-γ), 6 per group (TNF-α), 7 Ctr, 6 Saracatinib (IL-12 p40), and 6 per group (NOS2). Brain—n=5 per group (IFN-γ, TNF-α, IL-12 p40, NOS2). Statistical significance was determined using two-way unpaired t test. (H) Serum levels of IFN-γ, TNF-α and IL-12 p40 were measured by ELISA. Data shown as mean±SEM from mice pooled from 2 independent experiments: n=7 per group (IFN-γ), 5 Ctr, 7 Saracatinib (TNF-α), and 6 Ctr, 7 Saracatinib (IL-12 p40). Statistical significance was determined using two-way unpaired t test. (I) Serum titers of anti-T. gondii IgG were measured by ELISA and are represented as mean±SEM from 5 mice per group. Results are representative of 2 independent experiments. (J, K) B6 mice infected with ME49 T. gondii tissue cysts for 6 weeks were treated with Saracatinib or vehicle for four days. Brain sections were stained with antibodies against T. gondii, phospho-5473 Akt or PTEN. Scale bar, 10 m. Data is shown as mean±SEM from 4 mice. Statistical significance determined using two-way unpaired Student's t Test (*p<0.05, **p<0.01, ***p<0.001, ****p<0.0001).

FIGS. 7(A-B) illustrate graphs showing Saracatinib confers protection against ocular and cerebral toxoplasmosis in a Beclin 1-dependent manner. Becn1^{+/+} and Becn1^{+/-} mice were infected with 10 T. gondii ME49 tissues cysts. Beginning at 4 weeks post-infection, mice were treated with or without Saracatinib (10 mg kg⁻¹ oral gavage 2× per day; 5 days per week) for 2 weeks. (A) Representative images of retinas and brains as well as histopathological scoring. Retina: arrowhead=vitreal inflammation; arrow=perivascular inflammation; asterix=disruption of retinal architecture. Brain: arrowhead=perivascular inflammation; arrow, tissue cyst; asterix; microglial nodule. Scale bar, 50 m. Data are shown as Data are shown as mean±SEM from mice pooled from 2 independent experiments: n=5 (DMSO, Beclin1+/+), 6 (Saracatinib, Beclin1+/+), 3 (DMSO, Beclin1+/−), and 4 (Saracatinib, Beclin1+/−). Statistical significance was determined using two-way ANOVA with Tukey multiple comparison test. (B) T. gondii B1 gene was examined in the eye by qPCR. Levels were compared to those of one control mouse that was given an arbitrary value of 1. Number of T. gondii tissue cysts were counted in brain homogenates. Bars represent mean±SEM from mice pooled from 2 independent experiments: n=5 (DMSO, Beclin1+/+), 6 (Saracatinib, Beclin1+/+), 3 (DMSO, Beclin1+/−), and 3 or 4 (Saracatinib, Beclin1+/−). Statistical significance determined using one-way ANOVA with Tukey's multiple comparison test (*p<0.01,***p<0.001, ****p<0.0001).

FIGS. 8(A-B) illustrate schematics showing inhibition of Src signaling induces autophagic targeting of T. gondii via PTEN-mediated deactivation of Akt. T. gondii can activate Src independently of EGFR. (A) Activated Src associates with PTEN inducing phosphorylation of residues that deactivate PTEN. Src promotes PI3K and Akt activation. Activated Akt is recruited to the PVM and blocks autophagic targeting of the parasite. (B) Deficiency of Src or treatment with Saracatinib enables PTEN activation, recruitment of PTEN to the PVM and autophagic killing of T. gondii. These events occur both in EGFR+ and EGFR− cells. $ represents phosphorylation of residues indicative of activated molecules: phospho-Tyr 416 Src; phospho-Y199 PI3K, phospho-Ser473 Akt. ◯ represents phosphorylation of residues indicative of deactivated PTEN: phospho-Tyr240, phospho-Ser380 and phospho-Thr382/383. Figure generated with BioRender (with permission).

FIG. 9 illustrates graphs showing the assessment of potential cell toxicity of Saracatinib. CHO cells and retinal pigment epithelial cells (RPE) were treated with various concentrations of Saracatinib (1 nM-1000 μM) followed by MTT assay 24 hrs. after incubation with Saracatinib. The data are pooled from duplicate or triplicate independent samples obtained in 3 different experiments. Data points are demonstrated as circles, triangles, or squares corresponding to replicates within each experiment. Data are shown as mean±SEM. Significance was determined by comparing all groups to the control using one-way ANOVA with Holm-Sidak multiple comparison test (*p<0.05). (PDF)

FIGS. 10A-B illustrate images and graphs showing Saracatinib does not affect invasion of mammalian cells or intracellular replication of the parasite. (A) CHO cells were treated with Saracatinib (1 μM) and infected with RH-RFP T. gondii for 2 hours. Cells were fixed with Paraformaldehyde (PFA) and were not permeabilized prior to staining with a tachyzoite marker, SAG1. Intracellular cells will express red fluorescence while extracellular parasites stain for SAG1 and appear green or yellow in color. Data are shown as mean±SEM. The graphs show data from 7 independent monolayers pooled from 2 different experiments. (B) CHO cells were treated with Saracatinib (1 μM) and infected with RH-RFP T. gondii for 24 hours. Number of tachyzoites per vacuole were determined by light microscopy and quantified as percentage of total vacuoles. Data are shown as mean±SEM. The graphs show data from 6 independent monolayers pooled from 3 different experiments. No significant difference was found between control and Saracatinib-treated groups following statistical analysis using two-way, unpaired Student's t test. (PDF)

FIGS. 11(A-B) illustrate plots and assays showing Src but not Fyn, Yes or c-Abl regulate T. gondii survival. (A) Wildtype fibroblasts (MEF), MEFs deficient in Src/Yes/Fyn (SYF), or SYF MEFs reconstituted to express Src were infected with RH T. gondii. Percentage of infected cells and number of tachyzoites per 100 cells were determined by light microscopy. Data shown as mean±SEM. The graphs show data from 3 independent monolayers per group pooled from 3 different experiments. Significance for the percentages of infected cells was determined using two-way ANOVA with Tukey's multiple comparison test. Statistical analysis shown represents comparisons of 2 v. 24 hrs. Significance for tachyzoites per 100 cells was determined using one-way ANOVA with Dunnett's multiple comparison test comparing groups to wild-type (WT) control. (*p<0.05, ****p<0.0001). (B) CHO cells were treated with Imatinib, a c-Abl inhibitor (0.3 μM or 1 μM) and infected with RH-RFP T. gondii for 2 or 24 hours. Data are shown as mean±SEM from four independent monolayers per group pooled from 2 different independent experiments. No significant difference was found comparing all groups using one- or two-way ANOVA as described above. (PDF)

FIGS. 12(A-B) illustrate plots showing endothelial cells deficient in Src exhibit toxoplasmacidal activity that is regulated by ULK1 and Akt. (A) Mouse endothelial cells (mHEVc) transduced with control shRNA or Src shRNA were transfected with control siRNA or ULK1 siRNA followed by infection with RH RFP T. gondii. Data shown as mean±SEM. The graphs show data from 4 independent monolayers pooled from 2 different experiments. (B) mHEVc cells transduced with control shRNA or Src shRNA were transfected with a plasmid that encodes WT-Akt or CA-Akt followed by challenge with T. gondii. Monolayers were examined as above. All data are shown as mean±SEM. The graphs show data from 4 independent monolayers pooled from 2 different experiments. All significance was determined using two-way ANOVA with Dunnett's multiple comparison test comparing Ctr vs. Src shRNA in each condition. (*p<0.05, **p<0.01, ***p<0.001, ****p<0.0001). (PDF)

FIG. 13 illustrates tissue from B6 Mice express endogenous levels of Src protein expression. Western blot analysis of Src expression from brain, retina, lung, liver and spleen of healthy two B6 mice. 50 μg of each tissue lysate were loaded and probed for Src and Actin. Relative density of Src was normalized to total Actin and compared to the Brain Sample #1. Densitometry analysis of blots shown from two mice.

FIG. 14 illustrates a graph showing B6 mice develop cerebral toxoplasmosis at 4-weeks post-infection. B6 female mice aged 6-8 weeks-old were infected with 10 ME49 T. gondii via i.p. injection. Following 4 weeks of infection, mice were euthanized and assessed for parasite load in the brain by counting the number of tissue cysts found per brain. Data are shown as mean±SEM from 10 mice pooled from 2 independent experiments.

FIGS. 15(A-F) illustrate images and a graph showing Saracatinib treatment provides protection against ocular and cerebral toxoplasmosis in Dexamethasone-treated mice. Female B6 mice aged 6-8 weeks were infected with 10 ME49 tissue cysts for 3 weeks. Mice were then treated with Dexamethasone (0.1 mg kg-1, i. p injection) for 10-14 days before treatment with Saracatinib (10 mg kg-1 oral gavage 2× day, 5 days a week) for 2 weeks. (A-B) Representative images of retinas and brains treated with PBS or Dexamethasone as well as histopathological scoring. Retina: arrowhead=vitreal inflammation; arrow=perivascular inflammation; asterisk=disruption of retinal architecture. Brain: arrowhead=perivascular inflammation; arrow, tissue cyst; asterisk; microglial nodule. Scale bar, 50 m. Data represents mean±SEM from 8 mice per group pooled from 2 independent experiments. Statistical significance was determined using unpaired Student's t test comparing. (*p<0.05, **p<0.01, ***p<0.001). (C-D) Representative images of retinas and brains treated with Dexamethasone with or without Saracatinib treatment as well as histopathological scoring. Retina: arrowhead=vitreal inflammation; arrow=perivascular inflammation; asterisk=disruption of retinal architecture. Brain: arrowhead=perivascular inflammation; arrow, tissue cyst; asterisk; microglial nodule. Scale bar, 50 μm. Data are shown as mean±SEM from mice pooled from 2 independent experiments: Retina—n=13 (Dexamethasone) and 9 (Dexamethasone+Saracatinib). Brain—n=8 (Dexamethasone) and 10 (Dexamethasone+Saracatinib). Statistical significance was determined using unpaired Stu-dent's t test (***p<0.001, ****p<0.0001). (E) T. gondii B1 gene was examined in the eye using qPCR. Levels were compared to those of one control mouse that was given an arbitrary value of 1. Bars represent mean±SEM pooled from mice pooled from 2 independent experiments: n=5 (PBS), 7 (Dexamethasone), and 6 (Dexamethasone+Saracatinib). Statistical significance was determined one-way ANOVA with Tukey's multiple comparison test (**p<0.01). (F) Number of T. gondii tissue cysts were counted per brain homogenate. Data are shown as mean±SEM from mice pooled from 2 independent experiments: n=4 (PBS), 12 (Dexamethasone) and 10 (Dexamethasone+Saracatinib). Statistical significance was determined using one-way ANOVA with Tukey's multiple comparison test (***p<0.001).

### DETAILED DESCRIPTION

For convenience, certain terms employed in the specification, examples, and appended claims are collected here. Unless defined otherwise, all technical and scientific terms used herein have the same meaning as commonly understood by one of ordinary skill in the art to which this application belongs.

Unless otherwise specified or indicated by context, the terms “a”, “an”, and “the” mean “one or more.” For example, “a compound” should be interpreted to mean “one or more compounds.”

The terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “or” as used herein should be understood to mean “and/or”, unless the context clearly indicates otherwise.

The term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

The term “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

“Pharmaceutically acceptable excipient” and “pharmaceutically acceptable carrier” refer to a substance that aids the administration of an active agent to and absorption by a subject and can be included in the compositions described herein without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutical composition” refers to a formulation containing the disclosed compounds in a form suitable for administration to a subject. In a preferred embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient (e.g., a formulation of the disclosed compound or salts thereof) in a unit dose of composition is an effective amount and varies according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, intranasal, inhalational, and the like. Dosage forms for the topical or transdermal administration of a compound described herein includes powders, sprays, ointments, pastes, creams, lotions, gels, solutions, patches, nebulized compounds, and inhalants. In a preferred embodiment, the active compound is mixed under sterile conditions with a pharmaceutically acceptable carrier, and with any preservatives, buffers, or propellants that are required.

A “patient,” “subject,” or “host” to be treated by the subject method may mean either a human or non-human animal, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

The terms “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

A “subject in need of treatment” may include a subject, patient, or individual having or at risk for developing a disease, disorder, or condition that is associated with infection by an intracellular pathogen, such as T. gondii. A “subject in need of treatment” may include, for example, a subject, patient, or individual having or at risk for developing a disease, disorder, or condition that is associated with infection by T. gondii. For example, a “subject in need of treatment” may include a patient having or at risk for developing toxoplasmosis.

The term “small molecule” is an art-recognized term. In certain embodiments, this term refers to a molecule, which has a molecular weight of less than about 2000 amu, or less than about 1000 amu, and even less than about 500 amu.

All percentages and ratios used herein, unless otherwise indicated, are by weight.

Embodiments described herein relate to compositions and methods of inducing killing and/or degradation of an intracellular pathogen, such as T. gondii, in a subject in need thereof, and particularly relate to compositions and methods of treating toxoplasmosis, such as ocular, neural, and/or congenital toxoplasmosis, as well as retinitis and encephalitis caused by intracellular pathogen infection, e.g., toxoplasmosis, in a subject in need thereof.

T. gondii, a causative agent of retinitis and encephalitis, survives within host cells by avoiding autophagy-dependent degradation by the lysosome. While T. gondii activates EGFR to avoid autophagic killing, EGFR expression is limited in neural tissue. We found that, independently of EGFR, T. gondii activates the ubiquitous molecule, Src, resulting in PTEN inhibition and decoration of the parasite-containing vacuole with activated Akt (negative regulator of autophagy). Inhibition of Src, by Src knockdown or treatment with an Src family kinase inhibitor, impaired this cascade, leading to autophagic killing of T. gondii demonstrated by autophagosomal and lysosomal marker recruitment and parasite killing dependent on ULK1 and lysosomal enzymes. Treatment with the Src family kinase inhibitor induced PTEN recruitment around parasites in neural tissue and impaired recruitment of activated Akt, causing a striking reduction in parasite load and histopathology in mice with ocular and cerebral toxoplasmosis. Autophagy-deficient mice treated with an Src family kinase inhibitor did not have improved parasite load or histopathology supporting the pivotal role of this pathway for parasite survival and development of toxoplasmosis.

In some embodiments, a method of inducing killing and/or degradation of an intracellular pathogen, such as an T. gondii, which activates Src and AKT to avoid killing and/or degradation of the intracellular pathogen, can include administering to the subject in need of treatment an amount of an Src family kinase inhibitor effective to inhibit activity, signaling, and/or function of Src and/or pathogen activation of Src and AKT in the subject.

In some embodiments, the intracellular pathogen can include an Apicomplexa, such as Babesia spp., Plasmodium spp., Cryptosporidium parvum, Cryptosporidium hominis, Cyclospora cayetanensis, Isospora belli, Neospora caninum, or T. gondii.

In some embodiments, the Apicomplexa is a parasitic protozoan, such as T. gondii.

In some embodiments, the subject has toxoplasmosis, such as neural toxoplasmosis, ocular toxoplasmosis, cerebral toxoplasmosis and/or congenital toxoplasmosis.

In other embodiments, the subject has retinitis and/or encephalitis caused by ocular and/or cerebral toxoplasmosis, and the administration of the Src family kinase inhibitor can be used to treat the retinitis and/or encephalitis.

The activity, signaling, and/or function of Src and/or pathogen activation of Src and AKT in the subject can be suppressed, inhibited, and/or blocked in several ways including: direct inhibition of the activity or expression of the Src family kinases (e.g., by using small molecules, peptidomimetics, dominant negative polypeptides, interfering RNA, gene therapy); activation of genes and/or proteins that inhibit one or more of, the activity, signaling, and/or function of the Src family kinases (e.g., by increasing the expression or activity of the genes and/or proteins); inhibition of genes and/or proteins that are downstream mediators of the Src family kinases (e.g., by blocking the expression and/or activity of the mediator genes and/or proteins); introduction of genes and/or proteins that negatively regulate one or more of, activity, signaling, and/or function of Src family kinases (e.g., by using recombinant gene expression vectors, recombinant viral vectors or recombinant polypeptides); or gene replacement with, for instance, a hypomorphic mutant of the Src family kinases (e.g., by homologous recombination, overexpression using recombinant gene expression or viral vectors, or mutagenesis).

In some embodiments, the Src family kinase inhibitor administered to the subject can include any drug or compound, such as a pharmacologic chemical species, a complex, peptide agent, fusion protein, or oligonucleotide that inhibits activity, signaling, and/or function of Src and/or pathogen activation of Src and AKT.

In some embodiments, the Src family kinase inhibitors can include a small molecule inhibitor of Src family kinases. Examples of small molecule Src family kinase inhibitors include at least one of N-(2-chloro-6-methylphenyl)-2-[[6-[4-(2-hydroxyethyl)-1-piperazinyl]-2-methyl-4-pyrimidinyl]amino]-5-thiazole carboxamide monohydrate (Dasatinib), N-(5-chloro-1,3-benzodioxol-4-yl)-7-(2-(4-methylpiperazin-1-yl)ethoxy)-5-(tetrahydro-2H-pyran-4-yloxy)quinazolin-4-amine (Saracatinib), 4-[(2,4-dichloro-5-methoxyphenyl)amino]-6-methoxy-7-[3-(4-methylpiperazin-1-yl)propoxy]quinoline-3-carbonitrile (bosutinib), (4-Amino-5-(4-methylphenyl)-7-(t-butyl)pyrazolo[3,4-d]-pyrimidine), (4-chlorophenyl)-7-(t-butyl)pyrazolo[3,4-d]-pyrimidine) (PP1), 1-tert-butyl-3-(4-chlorophenyl)pyrazolo[3,4-d]pyrimidin-4-amine (PP2), 6-(2,6-dichlorophenyl)-2-{[3-(hydroxymethyl)phenyl]amino}-8-methyl-7H,8H-pyrido[2,3-d]pyrimidin-7-one (PD1663266), (E)-N-[4-[3-chloro-4-(pyridin-2-ylmethoxy)anilino]-3-cyano-7-ethoxyquinolin-6-yl]-4-(dimethylamino)but-2-enamide (Neratinib), 3-(2-imidazo[1,2-b]pyridazin-3-ylethynyl)-4-methyl-N-[4-[(4-methylpiperazin-1-yl)methyl]-3-(trifluoromethyl)phenyl]benzamide (Ponatinib), (E)-N-[4-(3-chloro-4-fluoroanilino)-3-cyano-7-ethoxyquinolin-6-yl]-4-(dimethylamino)but-2-enamide (pelitinib), N-benzyl-2-[5-[4-(2-morpholin-4-ylethoxy)phenyl]pyridin-2-yl]acetamide (Tirbanibulin), 4-methyl-3-[(2-methyl-6-pyridin-3-ylpyrazolo[3,4-d]pyrimidin-4-yl)amino]-N-[3-(trifluoromethyl)phenyl]benzamide (NVP-BHG712), (2S,3S)-2,3-dihydroxybutanedioic acid; 6-(4-methylpiperazin-1-yl)-N-(5-methyl-1H-pyrazol-3-yl)-2-[(E)-2-phenylethenyl]pyrimidin-4-amine (ENMD-2076), 4-[4-[(5-tert-butyl-2-quinolin-6-ylpyrazol-3-yl)carbamoylamino]-3-fluorophenoxy]-N-methylpyridine-2-carboxamide (Rebastinib), analogues thereof, or any combination thereof.

In some embodiments, the small molecule Src family kinase inhibitor is N-(2-chloro-6-methylphenyl)-2-[[6-[4-(2-hydroxyethyl)-1-piperazinyl]-2-methyl-4-pyrimidinyl]amino]-5-thiazole carboxamide monohydrate (Dasatinib), N-(5-chloro-1,3-benzodioxol-4-yl)-7-(2-(4-methylpiperazin-1-yl)ethoxy)-5-(tetrahydro-2H-pyran-4-yloxy)quinazolin-4-amine (Saracatinib), or an analogue thereof.

The Src family kinase inhibitor may be administered in any useful concentration to a subject that has an intracellular pathogen infection, e.g., T. gondii infection, toxoplasmosis, such as ocular, neural, cerebral, and/or congenital toxoplasmosis, as well as retinitis and encephalitis caused by toxoplasmosis. For example, an Src family kinase inhibitor may be administered to the subject at a concentration of about 0.5 nanomolar (nM), about 1 nM, about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 micromolar (μM), about 2 μM, about 3 μM, about 4 μM, about 5 μM, about 6 μM, about 7 μM, about 8 μM, about 9 μM, about 10 μM.

In some embodiments, an Src family kinase inhibitor may be administered at a concentration of at least about 0.5 nanomolar (nM), at least about 1 nM, at least about 10 nM, at least about 20 nM, at least about 30 nM, at least about 40 nM, at least about 50 nM, at least about 60 nM, at least about 70 nM, at least about 80 nM, at least about 90 nM, at least about 100 nM, at least about 200 nM, at least about 300 nM, at least about 400 nM, at least about 500 nM, at least about 600 nM, at least about 700 nM, at least about 800 nM, at least about 900 nM, at least about 1 micromolar (μM), at least about 2 μM, at least about 3 μM, at least about 4 μM, at least about 5 μM, at least about 6 μM, at least about 7 μM, at least about 8 μM, at least about 9 μM, at least about 10 μM.

In other embodiments, a small molecule Src family kinase inhibitor may be administered at a concentration of at most about 10 μM, at most about 9 μM, at most about 8 μM, at most about 7 μM, at most about 6 μM, at most about 5 μM, at most about 4 μM, at most about 3 μM, at most about 2 μM, at most about 1 μM, at most about 900 nM, at most about 800 nM, at most about 700 nM, at most about 600 nM, at most about 500 nM, at most about 400 nM, at most about 300 nM, at most about 200 nM, at most about 100 nM, at most about 90 nM, at most about 80 nM, at most about 70 nM, at most about 60 nM, at most about 50 nM, at most about 40 nM, at most about 30 nM, at most about 20 nM, at most about 10 nM, at most about 1 nM, at most about 0.5 nM, etc. A range of concentrations may be used, e.g., between 22 nM-1 μM). Where more than one small molecule is used, the concentrations may be the same or different for each small molecule used.

The Src family kinase inhibitor may be administered in any useful dose. In some embodiments, the useful dose of the Src family kinase inhibitor is a therapeutic dose effective to treat a subject that has an intracellular pathogen infection, e.g., T. gondii infection, toxoplasmosis, such as ocular, neural, cerebral, and/or congenital toxoplasmosis, as well as retinitis and encephalitis caused by toxoplasmosis or a subtherapeutic dose that is below what is used for treating a disease, such as a T. gondii infection, toxoplasmosis, retinitis and encephalitis caused by toxoplasmosis, or for producing an optimal therapeutic effect by administration of the Src family kinase inhibitor alone to the subject.

For example, a small molecule Src family kinase inhibitor may be administered at a dose of about 50 micrograms (g), a dose of about 100 μg, a dose of about 200 μg, a dose of about 300 μg, a dose of about 400 μg, a dose of about 500 μg, a dose of about 750 μg, a dose of about 1 milligram (mg), a dose of about 1.2 mg, a dose of about 1.5 mg, a dose of about 2 mg, a dose of about 3 mg, a dose of about 4 mg, a dose of about 5 mg, a dose of about 6 mg, a dose of about 8 mg, a dose of about 10 mg, a dose of about 12 mg, a dose of about 15 mg, a dose of about 20 mg, a dose of about 25 mg, a dose of about 30 mg, a dose of about 40 mg, a dose of about 50 mg, a dose of about 60 mg, a dose of about 80 mg, a dose of about 100 mg, a dose of about 120 mg, a dose of about 140 mg, a dose of about 160 mg, a dose of about 180 mg, a dose of about 200 mg, a dose of about 225 mg, a dose of about mg, a dose of about 250 mg, a dose of about 275 mg, a dose of about 300 mg, a dose of about 350 mg, a dose of about 400 mg, a dose of about 500 mg, a dose of about 600 mg, a dose of about 800 mg.

In some embodiments, the small molecule Src family kinase inhibitor may be administered in any useful dose. For example, a small molecule Src family kinase inhibitor may be administered at a dose of at least 50 micrograms (μg), a dose of at least 100 μg, a dose of at least 200 μg, a dose of at least 300 μg, a dose of at least 400 μg, a dose of at least 500 μg, a dose of at least 750 μg, a dose of at least 1 milligram (mg), a dose of at least 1.2 mg, a dose of at least 1.5 mg, a dose of at least 2 mg, a dose of at least 3 mg, a dose of at least 4 mg, a dose of at least 5 mg, a dose of at least 6 mg, a dose of at least 8 mg, a dose of at least 10 mg, a dose of at least 12 mg, a dose of at least 15 mg, a dose of at least 20 mg, a dose of at least 25 mg, a dose of at least 30 mg, a dose of at least 40 mg, a dose of at least 50 mg, a dose of at least 60 mg, a dose of at least 80 mg, a dose of at least 100 mg, a dose of at least 120 mg, a dose of at least 140 mg, a dose of at least 160 mg, a dose of at least 180 mg, a dose of at least 200 mg, a dose of at least 225 mg, a dose of at least mg, a dose of at least 250 mg, a dose of at least 275 mg, a dose of at least 300 mg, a dose of at least 350 mg, a dose of at least 400 mg, a dose of at least 500 mg, a dose of at least 600 mg, a dose of at least 800 mg.

In other embodiments, the small molecule Src family kinase inhibitor may be administered at a dose of at most 800 mg, at a dose of at most 600 mg, at a dose of at most 500 mg, at a dose of at most 400 mg, at a dose of at most 300 mg, at a dose of at most 250 mg, at a dose of at most 225 mg, at a dose of at most 200 mg, at a dose of at most 180 mg, at a dose of at most 160 mg, at a dose of at most 140 mg, at a dose of at most 120 mg, at a dose of at most 100 mg, at a dose of at most 80 mg, at a dose of at most 60 mg, at a dose of at most 50 mg, at a dose of at most 40 mg, at a dose of at most 30 mg, at a dose of at most 25 mg, at a dose of at most 20 mg, at a dose of at most 15 mg, at a dose of at most 12 mg, at a dose of at most 10 mg, at a dose of at most 8 mg, at a dose of at most 6 mg, at a dose of at most 5 mg, at a dose of at most 4 mg, at a dose of at most 3 mg, at a dose of at most 2 mg, at a dose of at most 1.5 mg, at a dose of at most 1.2 mg, a dose of at most 1 mg, a dose of at most 750 μg, a dose of at most 600 μg, a dose of at most 500 μg, a dose of at most 400 μg, a dose of at most 350 μg, a dose of at most 300 μg, a dose of at most 250 μg, a dose of at most 200 μg, a dose of at most 180 μg, a dose of at most 150 g, a dose of at most 120 μg, a dose of at most 100 μg, a dose of at most 80 μg, a dose of at most 50 μg, a dose of at most 20 μg, a dose of at most 10 μg.

In some embodiments, where more than one small molecule Src family kinase inhibitor is used, the dosages may be the same or different for each small molecule Src family kinase inhibitor used. Where more than one small molecule is used, the dosing frequencies may be the same or different for each molecule used.

In some embodiments, an Src family kinase inhibitor, such as dasatinib and/or saracatinib, can be administered to the subject at a dose of less than about 150 mg per day, less than about 140 mg per day, less than about 130 mg per day, less than about 120 mg per day, less than about 110 mg per day, less than about 100 mg per day, less than about 90 mg per day, less than about 80 mg per day less than about 70 mg per day, less than about 60 mg per day, or less than about 50 mg per day.

In some embodiments, an Src family kinase inhibitor, such as dasatinib and/or saracatinib, can be administered at an amount effective to provide a plasma drug level less than about 250 ng/ml, less than about 200 ng/ml, less than about 150 ng/ml, or less than about 100 ng/ml.

In other embodiments, the one or more Src family kinase inhibitor can be an agent that causes a decrease in expression or activity of Src. The agent can include a nucleic acid molecule, e.g., an interfering RNA molecule. The RNA molecule can include any suitable RNA molecule and size sufficient to decrease the expression level or activity of Src. The RNA molecule may comprise a small hairpin RNA (shRNA) molecule, a small interfering RNA (siRNA), a microRNA (miRNA), or other useful RNA molecule. In some examples, the RNA molecule may comprise a messenger RNA (mRNA), transfer RNA (tRNA), ribosomal RNAs (rRNA), small nuclear RNA (snRNA), piwi-interacting (piRNA), non-coding RNA (ncRNA), long non-coding RNA, (lncRNA), and fragments of any of the foregoing. The RNA molecule may be single-stranded, double-stranded, or partially single- or double-stranded.

It will be appreciated that one or more Src family kinase inhibitors (e.g., small molecules and RNA molecules) are listed as examples and that a combination of Src family kinase inhibitor types may be used to treat the subject. For instance, administering one or more different types of Src family kinase inhibitors may be used to decrease the expression or activity of Src. For example, a small molecule Src family kinase inhibitor may be co-administered with an RNA molecule. These combinations are non-limiting examples of different combinations of Src family kinase inhibitors that may be used to treat the subject having or suspected of having an intracellular pathogen infection, such as toxoplasmosis.

While it may be possible to administer the Src family kinase inhibitor disclosed herein alone for administration to a subject having or suspected of having an intracellular pathogen infection, such as T. gondii infection, e.g., toxoplasmosis, or retinitis and encephalitis caused by toxoplasmosis, the Src family kinase inhibitor normally will be present as an active ingredient in a pharmaceutical composition. The pharmaceutical composition can include a therapeutically effective amount of one or more of the Src family kinase inhibitors. The therapeutically effective amount of an Src family kinase inhibitor described herein can depend on the route of administration, the type of mammal that is the subject, and the physical characteristics of the subject being treated. Specific factors that can be taken into account include disease severity and stage, weight, diet and concurrent medications. The relationship of these factors to determining a therapeutically effective amount of the disclosed compounds is understood by those of ordinary skill in the art.

The pharmaceutical compositions may be formulated in conventional manner, together with other pharmaceutically acceptable excipients if desired, into forms suitable, for example, for oral, parenteral, or topical administration. The modes of administration may include parenteral, for example, intramuscular, subcutaneous and intravenous administration, oral administration, topical administration and direct administration to sites of infection such as intraocular, intraaural, intrauterine, intranasal, intramammary, intraperitoneal, intralesional, etc.

Typically, oral administration or administration intravenously, such as via injection is preferred. However the particular mode of administration employed may be dependent upon the particular disease, condition of patient, toxicity of compound and other factors as will be recognized by a person of ordinary skill in the art.

Pharmaceutical compositions formulated for oral administration can include traditional inactive ingredients to provide desirable color, taste, stability, buffering capacity, dispersion, or other known desirable features. Examples include red iron oxide, silica gel, sodium laurel sulphate, titanium dioxide, edible white ink, and the like. Conventional diluents may be used to make compressed tablets. Both tablets and capsules may be manufactured as sustained-release compositions for the continual release of medication over a period of time. Compressed tablets may be in the form of sugar coated or film coated tablets, or enteric-coated tablets for selective disintegration in the gastrointestinal tract. Liquid dosage forms for oral administration may contain coloring and/or flavoring to increase patient compliance. As an example, the oral formulation comprising compounds of the invention may be a tablet comprising any one, or a combination of, the following excipients: calcium hydrogen phosphate dehydrate, microcrystalline cellulose, lactose, hydroxypropyl methyl cellulose, and talc.

The pharmaceutical compositions described herein may be in the form of a liquid formulation. Examples of preferred liquid compositions include solutions, emulsions, injection solutions, solutions contained in capsules. The liquid formulation may comprise a solution that includes a therapeutic agent dissolved in a solvent. Generally, any solvent that has the desired effect may be used in which the therapeutic agent dissolves and which can be administered to a subject. Generally, any concentration of therapeutic agent that has the desired effect can be used. The formulation in some variations is a solution which is unsaturated, a saturated or a supersaturated solution. The solvent may be a pure solvent or may be a mixture of liquid solvent components. In some variations the solution formed is an in situ gelling formulation. Solvents and types of solutions that may be used are well known to those versed in such drug delivery technologies.

The pharmaceutical composition described herein may be in the form of a liquid suspension. The liquid suspensions may be prepared according to standard procedures known in the art. Examples of liquid suspensions include micro-emulsions, the formation of complexing compounds, and stabilizing suspensions. The liquid suspension may be in undiluted or concentrated form. Liquid suspensions for oral use may contain suitable preservatives, antioxidants, and other excipients known in the art functioning as one or more of dispersion agents, suspending agents, thickening agents, emulsifying agents, wetting agents, solubilizing agents, stabilizing agents, flavoring and sweetening agents, coloring agents, and the like. The liquid suspension may contain glycerol and water.

The pharmaceutical composition described herein may be in the form of topical preparations. The topical preparation may be in the form of a lotion or a cream, prepared using methods known in the art. For example, a lotion may be formulated with an aqueous or oily base and may include one or more excipients known in the art, functioning as viscosity enhancers, emulsifying agents, fragrances or perfumes, preservative agents, chelating agents, pH modifiers, antioxidants, and the like. For example, the topical formulation comprising one or more compounds of the invention may be a gel comprising anyone, or a combination of, the following excipients: PEG 8000, PEG 4000, PEG 200, glycerol, propylene glycol. The NCL812 compound may further be formulated into a solid dispersion using SoluPlus (BASF, www.soluplus.com) and formulated with anyone, or a combination of, the following excipients: PEG 8000, PEG 4000, PEG 200, glycerol, and propylene glycol.

For aerosol administration, the pharmaceutical composition can be provided in a finely divided form together with a non-toxic surfactant and a propellant. The surfactant is preferably soluble in the propellant. Such surfactants may include esters or partial esters of fatty acids.

The pharmaceutical compositions may alternatively be formulated for delivery by injection. As an example, the Src family kinase inhibitor can be delivered by injection by any one of the following routes: intravenous, intramuscular, intradermal, intraperitoneal, and subcutaneous.

The pharmaceutical compositions may alternatively be formulated using nanotechnology drug delivery techniques such as those known in the art. Nanotechnology-based drug delivery systems have the advantage of improving bioavailability, patient compliance and reducing side effects

The Src family kinase inhibitors described herein may be dissolved in oils/liquid lipids and stabilized into an emulsion formulation. Nanoemulsions may be prepared using high- and low-energy droplet reduction techniques. High-energy methods may include high-pressure homogenization, ultrasonication and microfluidisation. If the low-energy method is used, solvent diffusion and phase inversion will generate a spontaneous nanoemulsion. Lipids used in nanoemulsions may be selected from the group comprising triglycerides, soybean oil, safflower oil, and sesame oil. Other components such as emulsifiers, antioxidants, pH modifiers and preservatives may also be added.

The composition may be in the form of a controlled-release formulation and may include a degradable or non-degradable polymer, hydrogel, organogel, or other physical construct that modifies the release of the compound. It is understood that such formulations may include additional inactive ingredients that are added to provide desirable color, stability, buffering capacity, dispersion, or other known desirable features. Such formulations may further include liposomes, such as emulsions, foams, micelles, insoluble monolayers, liquid crystals, phospholipid dispersions, lamellar layers, and the like. Liposomes for use in the invention may be formed from standard vesicle-forming lipids, generally including neutral and negatively charged phospholipids and a sterol, such as cholesterol.

The Src family kinase inhibitors are particularly effective when administered in combination with one or more other agents or therapies useful in the treatment of intracellular pathogen infections. For example, one or more Src family kinase inhibitor can be administered in combination with effective doses of other medicinal and pharmaceutical agents, or in combination with other non-medicinal therapies. The term “administration in combination with” refers to both concurrent and sequential administration of the active agents.

In some embodiments, the one or more Src family kinase inhibitors can be administered to the subject in combination with an antimicrobial agent and/or an EGFR inhibitor. The killing and/or degradation effect of the Src family kinase inhibitor on the pathogen can be enhanced as compared to the effect of the Src family kinase inhibitor administered without the antimicrobial agent.

The antimicrobial agent can include, for example, an antibiotic agent, antiprotozoal agent, and/or antiparasitic agent. In some embodiments, the antimicrobial agent can include at least one of

In some embodiments, the antimicrobial agent can include at least one of 4-aminoquinoline Amodiaquine, chloroquine, hydroxychloroquine, piperaquine (bis-4-aminoquinoline) 8-aminoquinoline Bulaquine, pamaquine, Primaquine, tafenoquine Acetamide Thiolutin Acridine dye Acriflavine, mepacrine (quinacrine) Alkylphosphocholine Miltefosine Allylamine Terbinafine Aminoglycosides Paromomycin Aminophenanthridium Homidium, isometamidium chloride, Aminopyridine MMV390048 antimalarials Antimonials, Sodium stibogluconate, pentavalent meglumine antimoniate Arsenicals Acetarsol (5+), arsthinol (trivalent & (3+), carbarsone (5+), pentavalent) difetarsone (5+), glycobiarsol (5+), melarsomine (3+), melarsoprol (3+), nitarsone (5+), oxophenarsine (3+), roxarsone (5+), tryparsamide (5+) Arylaminoalcohol Halofantrine, lumefantrine, quinine/quinidine Azo naphthalene dyes Trypan blue, trypan red Azoles (triazoles Albaconazole, itraconazole, and imidazoles) ketoconazole, posoconazole, ravuconazole Benzamide Zoxamide Benzenediol Resveratrol Benzimidazoles and Albendazole, fenbendazole, probenzimidazoles febantel, mebendazole, omeprazole Bicyclohexylammonium Fumagillin Carbamate Disulfiram Cinnamamido adenosine Puromycin Coumarin Flocoumafen Diamidines Amicarbalide, diminazene diaceturate, imidocarb dipropionate, pafuramidine, pentamidine isethionate, phenamidine isethionate, propamidine, stilbamidine Dichloroacetamide Clefamide, Etofamide, Teclozan Dichloroacetamide Diloxanide furoate Difluoromethylornithine Eflornithine Dihydrofolate Diaveridine, ormetoprim, reductase/thymidyate pyrimethamine, trimethoprim synthase inhibitors Dihydrooratate dehydrogenase (DHODH) inhibitors Dinitroaniline Trifluralin, oryzalin Dinitrocarbanilide+Nicarbazin pyrimidinol Dithiocarbamate Thiram Ethoxybenzoic acid Ethopabate Fluoroquinolones Ciprofloxacin, enrofloxacin, marbofloxacin Guanidines Chloroproguanil, cycloguanil, lauroguadine, proguanil, Robenidine Halogenated Iodoquinol, chlorquinaldol, 8-hydroxyquinoline tilbroquinol, broxyquinoline, diiodohydroxyquinoline, clioquinol Hydroxyoxo-Sethoxydim, tralkoxydim, cyclohexenecarbaldehyde alloxydim, clethodim and oxime cycloxydim Hydroxyquinolones Buquinolate, decoquinate, nequinate Imidazolopiperazine Kaf156 Isoquinoline Emetine/dehydroemetine Lincosamides Clindamycin, lincomycin Macrolides Azithromycin, clarithromycin, erythromycin, roxithromycin, spiramycin Methylquinolinium Quinapyramine, quinuronium sulfate Miscellaneous Pyridaben Naphthoquinones Atovaquone, buparvaquone, parvaquone Naphthyridine Pyronaridine Nitrobenzamides Aklomide, dinitolmide Nitrofurans Nifurtimox, furaltodone, furazolidone, nifuratel, nifuroxime, nifursol Nitroimidazoles Azanidazole, benznidazole, carnidazole, dimetridazole, fexnidazole, ipronidazole, metronidazole, nimorazole, ornidazole, propenidazole, ronidazole, satranidazole, secnidazole, ternidazole, tinidazole Nitrothiazoles Nitazoxanide, aminitrozole (nithiamide), forminitrazole, niridazole, tenonitrozole Organometallic Auranofin, ferroquine antiprotozoal Oxaborole (including SCYX-7158 benzoxaboroles) Phenoxyphenol Triclosan phenylsulfamide Tolylfluanid Phosphonic acid Fosmidomycin derivative Phosphonomethylglycine Glyphosate Phosphoramidothioic Amiprofos-methyl acid Polyene Amphotericin B, mepartricin, hachimycin, Hamycin Polyether ionophores Laidlomycin, lasalocid, maduramicin, monensin, narasin, salinomycin, semduramicin Polypeptide Bacitracin (zinc, methylene disalicylate), cecropins, cyclosporins, dermaseptin, magainins, tachyplesin, thiostrepton Polysulfonated Suramin naphthylamine Propylphosphonic acid Fosmidomycin Purinamine Arprinocid Pyrazolopyran Pyrazolopyrimidine Allopurinol Pyridinols Clopidol Pyrimidine Fenarimol Pyrrolidinediol Anisomycin Quinazolinone Febrifugine, halofuginone Quinoline Mefloquine, nequinate (methyl benzoquate), quinfamide, tiliquinol Quinoxaline Carbadox Rifamycin Rifaximin Spiroindolone KAE609 (formerly NJTD609), cipargamin Strobilurin Fluacrypyrim, azoxystrobin, trifloxystrobin, dimoxystrobin Sulfonamides Sulfadiazine, sulfadimethoxine, sulfadoxine, sulfaguanidine, sulfamethazine (sulfadimidine), sulfamethoxazole, sulfanitran, sulfaquinoxaline, sulfamethoxypyrazine, cyazofamid Sulphone Dapsone Tetracyclines Chlortetracycline, doxycycline, oxytetracycline, tetracycline, tigecycline Tetraoxanes Thiamine analogs Amprolium Thiophenone Thiolactomycin Translation elongation DDD107498 factor 2 (eEF2) inhibitor Triazine Clazuril, diclazuril, ponazuril, toltrazuril Triazole Bitertanol Trioxane (sesquiterpene Artemether, artesunate, lactones, dihydroartemisinin, artemotil, artemisinins) artemisinin, arteether, artemisone Trioxolane (including Arterolane, OZ277, OZ439 ozonides).

In some embodiments, the antimicrobial agent is an antiprotozoal agent that can treat protozoan-related conditions. Antiprotozoal agents, which can be administered in combination with the Src family kinase inhibitor, include established antiprotozoal drugs, natural product-derived antiprotozoal drugs, apoptosis-promoting drugs often with a history of use as apoptosis-promoting agents, and known antiprotozoal natural products.

In some embodiment, the antiprotozoal agents can include atovaquone, diaminopyrimidines, especially amodiaquine, amphotericin, butoconazole, astemizole clindamycin, eflornithine, fumagillin, the 8-hydroxyquinolines, iodoquinol (diiodohydroxyquin), clioquinol (iodochlorhydroxyquin), the 2-nitroimidazoles, Etanidazole, Benznidazole fluoroquinolones, enoxacin, ciprofloxacin, doxycycline, melarsoprol, metronidazole, tinidazole, miltefosine, nifurtimox, nitazoxanide, paromomycin, pentamindine, sodium stibogluconate, antimony gluconate (SAG), and related antimonials, suramin, including the sodium salt, tinidazole, pyrimethamine, proguanil (chloroguanide), spiramycin, and sulfadoxine. Also useful are detergent and non-detergent spermicides that have additional anti-protozoal activity when used in topical formulations (Gupta G. Microbicidal spermicide or spermicidal microbicide? Eur J Contracept Reprod Health Care. 2005 December; 10(4):212-8).

Natural product derived antiprotozoal drugs include, but are not limited to, sesquiterpene lactones related to artemisinin from Artemisia annua, particularly artemisinin, dihydroartemisinin, artemether, artesunate, and further derivatives of artemisinin described in the literature (Haynes, 2006, From artemisinin to new artemisinin antimalarials: biosynthesis, extraction, old and new derivatives, stereochemistry and medicinal chemistry requirements. Curr Top Med. Chem. 6(5):509-37); quinolines like quinine derived from the bark of the South American chinchona tree, including alkaloids structurally related to quinine, quinine and quine-related quinolines, halofantrine, mefloquine, lumefantrine, amodiaquine, pyronaridine, piperaquine, chloroquine, hydroxychloroquine, napthoquine, primaquine, tafenoquine, amodiaquine and 4-aminoquinolines derived from the quinolines (Neill et al., 2006, Curr Top Med Chem. 6:479-507); other quinones including those extracted from Salvia prionitis, particularly salvicine and its derivatives (Qing C. et al, In vitro cytotoxicity of a salvicine, a novel diterpenoid quinone, Zhongguo Yao Li Xue Bao. 1999 April; 20(4):297-302); curcuminoids derived from curcumin, extracted from Curcuma domestica, including 6-gingerol and 6-paradol (Surh et al., 1999, J Environ Pathol Toxicol Oncol. 18:131-9); selected flavonoids and isoflavones, including, but not limited to, Genistein from soy, and derivatives from a number of plant sources including dehydrosilybin, silybin A and silybin B and isosilybin A and isosilybin B, and 8-(1; 1)-DMA-kaempferide (Tasdemir et al., 2006, Antimicrob Agents Chemother. 50:1352-64), luteolin, baicalein, dihydrobetulinic acid, quercetin, eriodictyol acid, lursolic acid, oleanolic acid; and triterpenes, particularly Ganoderic acid X, isolated from Ganoderma amboinense and triterpene rich extracts of Sapindus mukorossi known to have anti-trichomonal activity.

Apoptosis promoting antiprotozoal agents include, but are not limited to, artemisinin derivatives, atovaquone, chloroquine, iodoquinol (diiodohydroxyquin), clioquinol (iodochlorhydroxyquin), Jasmonic acid [3-oxo-2-(2-pentenyl)cyclopentaneacetic acid], methyl jasmonate[methyl 3-oxo-2-(2-pentenyl)cyclopentaneacetic acid], and cis-jasmone[3-methyl-2-(2-pentenyl)-2-cyclopenten-1-one], 3,3′-dihexyloxacarbocyanine iodide, sodium stibogluconate, extracts of Yucca schidigera, and curcumin. Apoptosis promoting agents include, but are not limited to, Pyrroloquinazolinediamine, Novobiocin, quercetin, cyclosporine, dihydrobetulinic acid, campothectins, especially topotecan, irinotecan, SN38 (the active metabolite of irinotecan), bortezimib, etoposide, quinones including salvicine, and anthracyclines including doxorubicin, daunorubicin, 4′-epirubicin, idarlibicin, and deoxydoxorubicin.

Antiprotozoal natural products include teas and extracts made from Artemisia annua, teas and extracts made from Curcuma domestica, extracts from garlic which include allicin and other thiosulfinates, root extracts of Uvaria chamae (Annonaceae) and Hippocratea africana (Hippocrateaceae), and root extracts of Homalium letestui. Also useful are extracts of Brasilian plants with demonstrated anti-malarial activity, including Vernonia brasiliana and Acanthospermum australe (Carvalho, 1991, Braz J Med Biol Res. 24(11):1113-23, and Botsaris A S. Plants used traditionally to treat malaria in Brazil: the archives of Flora Medicinal. J Ethnobiol Ethnomed. 2007 May 1; 3:18).

In some embodiments, the antiprotozoal agent administered in combination with the Src family kinase inhibitor can include amoxicillin, atovaquone, diaminopyrimidines, especially amodiaquine, amphotericin, proguanil (chlorguanide), chloroquine, clindamycin, eflornithine, furazolidone, a fluoroquinolone, such as ciprofloxacin or levofloxacin, or a third generation cephalosporin, such as ceftriaxone or cefixime, hydroxychloroquine, mefloquine, melarsoprol, metronidazole, minocycline, nifursemizone, nitazoxanide, ornidazole, paromycin sulfate, pentamidine, pyrimethamine, quinapyramine, ronidazole, tinidazole, spriramycin, sulfadiazine, sulfamethoxazole, trimethoprim, or combinations thereof.

In some embodiments, the EGFR inhibitor can include, for example, an antibody, such as Erbitutux (cetuximab, Imclone Systems Inc.) and ABX-EGF (panitumumab, Abgenix, Inc.). In another embodiment, the EGFR inhibitor is a small molecule that competes with ATP, such as Tarceva (erlotinib, OSI Pharmaceuticals), Iressa (gefitinib, Astra-Zeneca), tyrphostins described by Dvir, et al., J Cell Biol., 113:857-865 (1991); tricyclic pyrimidine compounds disclosed in U.S. Pat. No. 5,679,683; compound 6-(2,6-dichlorophenyl)-2-(4-(2-diethylaininoethoxy)phenylamino)-8-methyl-8H-pyrido(2,3-d)pyrimidin-7-one (known as PD166285) disclosed in Panek, et al., Journal of Pharmacology and Experimental Therapeutics 283, 1433-1444 (1997).

In some embodiments, the EGFR inhibitor is EGFR tyrosine kinase inhibitor.

In other embodiments, the EGFR inhibitor can include at least one of gefitinib (Iressa), erlotinib (Tarceva), afatinib (Gilotrif), dacomitinib (Vizimol), or osimertinib (Targrisso).

In some embodiments, the concentration of the antimicrobial agent and/or EGFR inhibitor administered in combination with the Src family kinase inhibitor can be any useful concentration, and in particular an amount effective to treat and intracellular pathogen infection, such as a T. gondii infection, toxoplasmosis, such as ocular, neural, cerebral, and/or congenital toxoplasmosis, as well as retinitis and encephalitis caused by toxoplasmosis. For example, an antimicrobial and/or EGFR inhibitor may be administered to the subject in combination with the Src family kinase inhibitor at a concentration of about 0.5 nanomolar (nM), about 1 nM, about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 micromolar (μM), about 2 μM, about 3 μM, about 4 μM, about 5 μM, about 6 μM, about 7 μM, about 8 μM, about 9 μM, about 10 μM.

In some embodiments, an antimicrobial agent and/or EGFR inhibitor may be administered to the subject in combination with the Src family kinase inhibitor at a concentration of at least about 0.5 nanomolar (nM), at least about 1 nM, at least about 10 nM, at least about 20 nM, at least about 30 nM, at least about 40 nM, at least about 50 nM, at least about 60 nM, at least about 70 nM, at least about 80 nM, at least about 90 nM, at least about 100 nM, at least about 200 nM, at least about 300 nM, at least about 400 nM, at least about 500 nM, at least about 600 nM, at least about 700 nM, at least about 800 nM, at least about 900 nM, at least about 1 micromolar (M), at least about 2 μM, at least about 3 μM, at least about 4 μM, at least about 5 μM, at least about 6 μM, at least about 7 μM, at least about 8 μM, at least about 9 μM, at least about 10 μM.

In some embodiments, an antimicrobial agent and/or EGFR inhibitor may be administered to the subject in combination with the Src family kinase inhibitor at a concentration of at most about 10 μM, at most about 9 μM, at most about 8 μM, at most about 7 μM, at most about 6 μM, at most about 5 μM, at most about 4 μM, at most about 3 μM, at most about 2 μM, at most about 1 μM, at most about 900 nM, at most about 800 nM, at most about 700 nM, at most about 600 nM, at most about 500 nM, at most about 400 nM, at most about 300 nM, at most about 200 nM, at most about 100 nM, at most about 90 nM, at most about 80 nM, at most about 70 nM, at most about 60 nM, at most about 50 nM, at most about 40 nM, at most about 30 nM, at most about 20 nM, at most about 10 nM, at most about 1 nM, at most about 0.5 nM, etc. A range of concentrations may be used, e.g., between 22 nM-1 μM. Where more than one antimicrobial agent and/or EGFR inhibitor is used, the concentrations may be the same or different for each antimicrobial used.

The antimicrobial agent and/or EGFR inhibitor can be administered in combination with the Src family kinase inhibitor at any useful dose. In some embodiments, the useful dose of the antimicrobial and/or EGFR inhibitor administered to the subject in combination with the Src family kinase inhibitor is a therapeutic dose effective to treat a subject that has an intracellular pathogen infection, e.g., T. gondii infection, toxoplasmosis, such as ocular, neural, and/or congenital toxoplasmosis, as well as retinitis and encephalitis caused by toxoplasmosis or a subtherapeutic dose that is below what is used for treating a disease, such as T. gondii infection, toxoplasmosis, retinitis and encephalitis caused by toxoplasmosis, or for producing an optimal therapeutic effect by administration of the Src family kinase inhibitor alone to the subject.

For example, an antimicrobial and/or EGFR inhibitor administered to the subject in combination with the Src family kinase inhibitor can be at a dose of about 50 micrograms (μg), a dose of about 100 μg, a dose of about 200 μg, a dose of about 300 μg, a dose of about 400 μg, a dose of about 500 μg, a dose of about 750 μg, a dose of about 1 milligram (mg), a dose of about 1.2 mg, a dose of about 1.5 mg, a dose of about 2 mg, a dose of about 3 mg, a dose of about 4 mg, a dose of about 5 mg, a dose of about 6 mg, a dose of about 8 mg, a dose of about 10 mg, a dose of about 12 mg, a dose of about 15 mg, a dose of about 20 mg, a dose of about 25 mg, a dose of about 30 mg, a dose of about 40 mg, a dose of about 50 mg, a dose of about 60 mg, a dose of about 80 mg, a dose of about 100 mg, a dose of about 120 mg, a dose of about 140 mg, a dose of about 160 mg, a dose of about 180 mg, a dose of about 200 mg, a dose of about 225 mg, a dose of about mg, a dose of about 250 mg, a dose of about 275 mg, a dose of about 300 mg, a dose of about 350 mg, a dose of about 400 mg, a dose of about 500 mg, a dose of about 600 mg, a dose of about 800 mg.

The antimicrobial agent and/or EGFR inhibitor administered to the subject in combination with the Src family kinase inhibitor can be administered in any useful dose. For example, an antimicrobial and/or EGFR inhibitor administered to the subject in combination with the Src family kinase inhibitor can be administered at a dose of at least 50 micrograms (g), a dose of at least 100 μg, a dose of at least 200 μg, a dose of at least 300 μg, a dose of at least 400 μg, a dose of at least 500 μg, a dose of at least 750 μg, a dose of at least 1 milligram (mg), a dose of at least 1.2 mg, a dose of at least 1.5 mg, a dose of at least 2 mg, a dose of at least 3 mg, a dose of at least 4 mg, a dose of at least 5 mg, a dose of at least 6 mg, a dose of at least 8 mg, a dose of at least 10 mg, a dose of at least 12 mg, a dose of at least 15 mg, a dose of at least 20 mg, a dose of at least 25 mg, a dose of at least 30 mg, a dose of at least 40 mg, a dose of at least 50 mg, a dose of at least 60 mg, a dose of at least 80 mg, a dose of at least 100 mg, a dose of at least 120 mg, a dose of at least 140 mg, a dose of at least 160 mg, a dose of at least 180 mg, a dose of at least 200 mg, a dose of at least 225 mg, a dose of at least mg, a dose of at least 250 mg, a dose of at least 275 mg, a dose of at least 300 mg, a dose of at least 350 mg, a dose of at least 400 mg, a dose of at least 500 mg, a dose of at least 600 mg, a dose of at least 800 mg.

In some embodiments, the antimicrobial and/or EGFR inhibitor administered to the subject in combination with the Src family kinase inhibitor can be administered at a dose of at most 800 mg, at a dose of at most 600 mg, at a dose of at most 500 mg, at a dose of at most 400 mg, at a dose of at most 300 mg, at a dose of at most 250 mg, at a dose of at most 225 mg, at a dose of at most 200 mg, at a dose of at most 180 mg, at a dose of at most 160 mg, at a dose of at most 140 mg, at a dose of at most 120 mg, at a dose of at most 100 mg, at a dose of at most 80 mg, at a dose of at most 60 mg, at a dose of at most 50 mg, at a dose of at most 40 mg, at a dose of at most 30 mg, at a dose of at most 25 mg, at a dose of at most 20 mg, at a dose of at most 15 mg, at a dose of at most 12 mg, at a dose of at most 10 mg, at a dose of at most 8 mg, at a dose of at most 6 mg, at a dose of at most 5 mg, at a dose of at most 4 mg, at a dose of at most 3 mg, at a dose of at most 2 mg, at a dose of at most 1.5 mg, at a dose of at most 1.2 mg, a dose of at most 1 mg, a dose of at most 750 μg, a dose of at most 600 μg, a dose of at most 500 μg, a dose of at most 400 μg, a dose of at most 350 μg, a dose of at most 300 μg, a dose of at most 250 μg, a dose of at most 200 μg, a dose of at most 180 μg, a dose of at most 150 μg, a dose of at most 120 μg, a dose of at most 100 μg, a dose of at most 80 μg, a dose of at most 50 μg, a dose of at most 20 μg, a dose of at most 10 μg. Where more than one antimicrobial agent and/or EGFR inhibitor is used, the dosages may be the same or different for each antimicrobial used. Where more than one antimicrobial and/or EGFR inhibitor is used, the dosing frequencies may be the same or different for each molecule used.

Given the synergistic effect of the coadministration of Src family kinase inhibitor with an antimicrobial agent and/or EGFR inhibitor, Src family kinase inhibitors and antimicrobial agents and/or EGFR inhibitor when administered in combination can be at amounts or doses to achieve a therapeutic effect that are substantially less (i.e., subtherapeutic dose or amount) than the amounts or doses that would be required to achieve a therapeutic effect if each compound was administered alone. Co-administration of the Src family kinase inhibitor in combination with the antimicrobial agent and/or EGFR inhibitor to the subject can also mitigate resistance to a single agent. Such resistance results either in the requirement for higher dosages of the drug and/or the renewed symptoms. Therefore, there is a practical upper limit to the amount that a subject can receive. However, if two or more agents are used in concert, the dosage of any single drug can be lowered. This is beneficial to the patient since using lower levels of therapeutic agents is generally safer for the patient. Thus, in some aspects, the compositions described herein can be administered to a subject at a subtherapeutic level.

In some embodiments, the amount of the Src family kinase inhibitor is subtherapeutic when administered in the absence of the antimicrobial agent and/or EGFR inhibitor. In other embodiments, the amount of the Src family kinase inhibitor is subtherapeutic when administered in combination with the antimicrobial agent and/or EGFR inhibitor. In still other embodiments, the amounts of Src family kinase inhibitor, antimicrobial agent, and/or EGFR inhibitor administered are subtherapeutic when the Src family kinase inhibitor, antimicrobial agent, and/or EGFR inhibitor are administered alone.

In some embodiments, the Src family kinase inhibitor can be administered either alone or in combination with another drug, such as an antimicrobial agent and/or EGFR inhibitor, to effect a curative treatment regimen. A curative dose can include a dose at which the intracellular pathogen is cleared for 28 days. Typically, the curative dose is administered over several days, such as for 1 to 10 days, such as over 1 to 7 days, such as from 2 to 5 days, for example, over 3 days. The curative dose can be administered once to several times daily, and typically is given in a once, twice, or three times daily dosage regimen.

In some embodiments, each constituent of the therapeutic combination can be administered using the same route or different routes. For example, the Src family kinase inhibitors disclosed herein may be administered by any suitable route in the form of a pharmaceutical composition adapted to such route and in a dose effective for the treatment intended. Each constituent of the therapeutic combination can, for example, be administered orally, mucosally, topically, transdermally, rectally, pulmonarily, parenterally, intranasally, intravascularly, intravenously, intraarterial, intraperitoneally, intrathecally, subcutaneously, sublingually, intramuscularly, intrasternally, intraocularly, intravitreally, or by infusion techniques, in dosage unit formulations containing conventional pharmaceutically acceptable excipients as disclosed herein.

In other embodiments, the Src kinase inhibitors can be used either alone or in combination with another drug, such as antimicrobial agent and/or EGFR inhibitor, in a method of treating an Apicomplexa infection (e.g., Babesia spp., Plasmodium spp., Cryptosporidium parvum, Cyclospora cayetanensis, Cryptosporidium hominis, Isospora belli, Neospora caninum, Sarcicystis neurona, or T. gondii). The method can include administering to a subject in need thereof an effective amount of an Src family kinase inhibitor described herein either alone or in combination with another drug, such as an antimicrobial agent and/or EGFR inhibitor.

In some embodiments, the Src family kinase inhibitor alone or in combination with the antimicrobial and/or EGFR inhibitor as described herein is used as a medicament. In embodiments, the medicament is useful for treating a disease caused by Apicomplexa (e.g., Babesia spp., Plasmodium spp., Cryptosporidium hominis, Cryptosporidium parvum, Cyclospora cayetanensis, Isospora belli, Neospora caninum, or T. gondii).

In some embodiments, the Apicomplexa infection is a systemic infection including the central nervous system, placenta, retina, and the brain. In some embodiments, the Apicomplexa infection is in the central nervous system. In some embodiments, the Apicomplexa infection is in the placenta. In some embodiments, the Apicomplexa infection is in the retina. In some embodiments, the Apicomplexa infection is in the brain. In some embodiments, a method includes preventing chronic Apicomplexa infection. In some embodiments, the method includes preventing reactivation of the Apicomplexa in tissue cysts. In some embodiments, the method includes reducing Apicomplexa survival, relative to a control (e.g., the absence of the Src family kinase inhibitor described herein). In some embodiments, the method includes reducing Apicomplexa proliferation, relative to a control (e.g., the absence of the Src family kinase inhibitor described herein). In some embodiments, the method includes reducing Apicomplexa egress from a cell, relative to a control (e.g., Apicomplexa egress in the absence of the Src family kinase inhibitor described herein). In some embodiments, the method includes reducing Apicomplexa invasion of a cell, relative to a control (e.g., Apicomplexa invasion in the absence of the Src family kinase inhibitor described herein). In embodiments, the method includes reducing Apicomplexa motility, relative to a control (e.g., Apicomplexa motility in the absence of the Src family kinase inhibitor described herein). In embodiments, the method includes reducing microneme (e.g., organelles involved in motility and invasion) protein release, relative to a control.

In some embodiments, the Apicomplexa is Babesia spp., Plasmodium spp., Cryptosporidium parvum, Cryptosporidium hominis, Cyclospora cayetanensis, Isospora belli, Neospora caninum, or Toxoplasma gondii. In embodiments, the Apicomplexa is Toxoplasma gondii.

In some embodiments, the Apicomplexa infection is a chronic infection (e.g., toxoplasmosis). In some embodiments, the method includes reducing or eliminating Apicomplexa tissue cysts present during chronic infection. In some embodiments, the Apicomplexa infection is an acute infection (e.g., toxoplasmosis). In some embodiments, the Apicomplexa infection is a congenital infection (e.g., toxoplasmosis). In some embodiments, the Apicomplexa infection is in a newborn (e.g., toxoplasmosis). In some embodiments, the Apicomplexa infection is in an infant (e.g., toxoplasmosis). In some embodiments, the method includes preventing chronic (e.g., recurring) Apicomplexa infection. Chronic toxoplasmosis has also been associated with bipolar disorder, obsessive-compulsive disorder, schizophrenia, and addiction. See Acta Psychiatr Scand. 2015 September; 132(3):161-79. doi: 10.1111/acps.12423. Epub 2015 Apr. 15. In embodiments, the Apicomplexa infection is in an HIV-positive subject. In embodiments, the Apicomplexa infection is in an immunocompromised subject.

In some embodiments, the Src kinase inhibitors described herein may be used as an adjunct to, or replacement of drugs, such as sulfonamides and pyrimethamine. In other embodiments, the Src family kinase inhibitors described may be used to treat not only the disease or disorder but may also be used to treat the symptoms and effects of the disease or disorder. For example, a subject afflicted by schizophrenia may exhibit symptoms such as delusions, anhedonia, and avolition.

In some embodiments, a method of treating an Apicomplexa associated disease includes administering to a subject in need thereof an effective amount of a Src family kinase inhibitor alone or in combination with an antimicrobial agent and/or EGFR inhibitor. In some embodiments, the disease is encephalitis. In some embodiments, the disease is retinitis. In some embodiments, the disease is schizophrenia. In some embodiments, the disease is toxoplasmosis (e.g., chronic toxoplasmosis, acute toxoplasmosis, congenital toxoplasmosis, or toxoplasmosis in a newborn infant). In some embodiments, the disease is toxoplasmic retinitis or encephalitis. In embodiments, the disease is ocular toxoplasmosis. In embodiments, the disease is schizophrenia. In some embodiments, the disease is bipolar disorder, obsessive-compulsive disorder, or addiction.

Still other embodiments described herein relate to a pharmaceutical composition or combination therapy that includes the Src family kinase inhibitor in combination with an antimicrobial agent and/or an EGFR inhibitor for use in treating an intracellular pathogen infection. The Src family kinase inhibitor, the antimicrobial agent, and/or EGFR inhibitor are administered at an amount effective to induce killing and/or degradation of the intracellular pathogen in a subject in need thereof.

In some embodiments, a pharmaceutical composition or combination therapy including the Src family kinase inhibitor, the antimicrobial agent, and the optional EGFR inhibitor can each be formulated at a subtherapeutic dose.

The following example is included to demonstrate preferred embodiments of the invention. It should be appreciated by those of skill in the art that the techniques disclosed in the example, which follow, represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

T. gondii causes PKCα/β-dependent sustained activation of the ubiquitous signaling molecule, Src. We show that Src plays a pivotal role in protecting T. gondii from autophagic killing in a manner that is independent of the presence of EGFR in host cells. T. gondii activated Src even in mammalian cells that lack EGFR or express DN EGFR mutant known to ablate EGFR signaling. In T. gondii-infected cells, Src associated with Phosphatase and Tensin Homolog (PTEN) which was maintained in an inactive state. This enabled sustained activation of Akt at the level of the PV membrane (PVM) and blockade of autophagic targeting. Src knockdown or treatment with Saracatinib, a potent and orally available Src kinase inhibitor, impaired Src-PTEN association, causing PTEN recruitment around the PV and impairing Akt activation at the level of the PVM, which resulted in killing of T. gondii. Treatment with Saracatinib in mice with pre-established ocular and cerebral toxoplasmosis revealed that the Src-PTEN-Akt cascade is of critical importance in vivo. Saracatinib caused a profound decrease in parasite load in the eye and brain, as well as marked reduction in histopathology that were dependent on the autophagy protein, Beclin 1. These studies identified a signaling mechanism utilized by T. gondii to ensure its survival that is not restricted to the presence of EGFR and indicate that targeting this pathway leads to a striking control of toxoplasmosis.

### Materials and Methods

### Ethics Statement

All studies were performed in accordance with regulations of the Guide for the Care and Use of Laboratory Animals and the National Institute of Health. The protocol received approval from the Institutional Animal Care and Use Committee (IACUC) of Case Western Reserve University (Protocol Number: 2015-0130).

### Mice

C57BL/6 mice (Jackson Laboratories, Bar Harbor, ME) or Becn 1^{+/-} (C57BL/6 background; gift from Beth Levine, University of Texas Southwestern, Dallas, TX) were bred at the Animal Resource Center at Case Western Reserve University. Female mice between the ages of 6 to 8 weeks old were infected with 10 cysts of the ME49 T. gondii for four weeks. In certain experiments, mice were euthanized at 4 weeks to evaluate severity of ocular and cerebral toxoplasmosis. Littermates were randomly assigned and treated with vehicle or Saracatinib (10 mg kg⁻¹ or 15 mg kg⁻¹; SelleckChem, Houston, TX) via oral gavage two times per day, 5 days a week for 4 weeks. Mice were also treated with trimethoprim-sulfamethoxazole (120:600 mg kg⁻¹; Dot Scientific Inc, Burton, MI and Sigma, St. Louis, MO). In certain experiments, mice received Dexamethasone (0.1 mg kg⁻¹, i.p injection) for 2 weeks followed by Saracatinib treatment and continued administration of Dexamethasone. For immunofluorescence studies, mice were infected with 10 ME49 cysts for 6 weeks before treatment with vehicle or Saracatinib (10 mg kg⁻¹) twice daily for 4 days. Becn 1^{+/+} and Becn 1^{+/-} mice were infected with 10 ME49 tissue cysts for 4 weeks followed by treatment with Saracatinib (10 mg kg-1) for 2 weeks. All T. gondii infection experiments were conducted in animal Biosafety Level 2 facilities.

T. gondii

T. gondii tachyzoites of the RH and PTG strains were used to infect mammalian cells. RFP− expressing RH T. gondii (a gift from Boris Striepen, University of Pennsylvania) and GFP− expressing PTG T. gondii (a gift from George Yap, Rutgers University) were used for this study. Tachyzoites were maintained in human foreskin fibroblasts as described.

Mammalian Cells and In Vitro Infection with T. gondii

Chinese Hamster Ovary (CHO; a gift from Cathleen Carlin, Case Western Reserve University), human retinal pigment epithelial (RPE) cells (ARPE-19; American Type Culture Collection, ATCC, Manassas, VA), and mouse endothelial cells (mHEVc) transduced with Src shRNA or control encoding lentiviral vectors were cultured in complete media. Mouse brain endothelial cells expressing WT or DN EGFR were isolated as described. Endothelial cells were cultured in medium containing Endothelial Cell Growth Supplement (ECGS; Sigma-Aldrich, St Louis, MO). Mammalian cells were infected with tachyzoites of the RH or PTG stains of T. gondii; RH strain was used unless otherwise indicated. Cells were fixed at 2 hours or 24 hours followed by staining using Hema 3 Manual Staining System (Fisherbrand, Hampton, NH). At least 100 to 200 cells per monolayer were counted by examining a minimum of 5 random areas per monolayer. The percentage of infected cells, tachyzoites and vacuoles per 100 cells, and tachyzoites per vacuole were counted using light microscopy. Three independent experiments were performed. Saracatinib, Gefitinib (LC Laboratories, Woburn, MA) and lysosomal inhibitors leupeptin and pepstatin (10 μM; Millipore Sigma, Burlington, MA) were used in specified experiments. Each cell monolayer was generated and infected independently.

### Transfection

Cells were transfected with mouse Src siRNA (target sequence: 5′ GGGAGAACCUGGUGUGCAAUU 3′) (SEQ ID NO: 1), mouse ULK1 siRNA (Ambion, Carlsbad, CA), mouse PTEN siRNA (target sequence: 5′ GUAUAGAGCGUGCAGAUAAUU 3′) (SEQ ID NO: 2), or control siRNA (Santa Cruz Biotechnology, Santa Cruz, CA) using TransIT-X2 Dynamic Delivery System (Mirus, Madison, WI).

### Histology

Brains and eyes were fixed in 10% formalin before paraffin embedding. Hematoxylin & eosin (H&E) and periodic acid Schiff hematoxylin (P.A.S.H) were used to stain sections from eyes and brains respectively. Brain and ocular histopathology were scored as described previously.

### Immunofluorescence

Mammalian cells were infected with RFP-T. gondii (RH) and fixed with 4% paraformaldehyde. Cells were stained with antibodies against LC3 (MBL International, Woburn, MA), LAMP1 (DSHB, Iowa City, IA), or GRA7 (gift from John Boothroyd, Stanford University, Stanford, CA) followed by a fluorescently conjugated secondary antibodies (Jackson ImmunoResearch Laboratories, West Grove, PA). LC3 and LAMP1 were evaluated for accumulation at 5- and 7 hours post-infection, respectively. Cells were stained with antibodies against PTEN (Santa Cruz Biotechnology, Dallas, TX) or anti-phospho S473 Akt (Novus, Centennial, CO). Frozen brain sections were stained with antibodies against PTEN (Novus, Centennial, CO), phospho S473 Akt, SAG-1 (Invitrogen, Waltham, MA) or T. gondii (Biogenex, Fremont, CA). Parasites were evaluated blindly in multiple random fields of view and 100 parasites were examined per group. In-out assays were performed as described using RFP-T gondii, anti-SAG-1 antibody (Invitrogen, Waltham, MA) and an Alexa Fluor 488-conjugated secondary antibody. Olympus FV1200 confocal microscope (Evident/Olympus, Waltham, MA) equipped with UPlanSApa 60× and 100× 1.45NA objectives was used to evaluate the slides.

Deconvolved images were generated with Huygens Essential 23.10 software (Scientific Volume Imaging, Hilversum, the Netherlands) using the Standard deconvolution profile in Deconvolution Express. Images were processed in Photoshop CC 19.1.1. using similar linear adjustments for all samples.

### Immunoblot

Membranes were probed with antibodies against Src, phospho-Y416 Src, Akt, phospho-S473 Akt, PTEN, phospho-5380/T382/T383 PTEN, PI3K, phospho-Y458/Y199 PI3K, ULK1, EGFR, HA tag (all from Cell Signaling, Danvers, MA), phospho-Y240 PTEN (Sigma) and Actin (Santa Cruz Biotechnologies) followed by incubation with secondary antibodies (Cell Signaling and Santa Cruz Biotechnologies). Three independent experiments were performed.

### Real-Time Quantitative PCR

Genomic DNA was isolated using a DNeasy isolation kit (Qiagen, Germantown, MD) which was used as the template to measure T. gondii B1 gene expression. Samples were normalized to L32. RNA was isolated then reverse transcribed using a RNeasy Plus Kit and a Quantitect Reverse Transcription kit (Qiagen), respectively. cDNA was used as a template to measure IFN-γ, IL-12 p40, TNF-α, and NOS2 which were normalized to 18S rRNA. In addition, cDNA was used to measure EGFR. Primers for EGFR were generated using the NCBI Primer-Blast (FWD: 5′ AACAGAGCCCCGAATGACTG 3′ (SEQ ID NO: 3), REV: 5′ CGTTTTCGGACAATGTGGCG 3′) (SEQ ID NO: 4). SYBR Green master mix was used to evaluate gene expression. Samples were run using StepOne Real-Time PCR system (Applied Biosystems, Waltham, MA). Relative gene expression was calculated using the ΔΔCt method. Two to three technical replicates were run per sample and data shown represent the average of each sample.

### ELISA

IFN-γ and IL-12 p40 were measured using ELISA kits from R&D (Minneapolis, MN). TNF-α was measured using a kit from eBioscience (San Diego, CA). Anti-T. gondii IgG was detected by ELISA.

### Statistics

Experimental results were analyzed for statistical significance using a two-tailed, unpaired Student's t-test, one-way ANOVA, or two-way ANOVA with Tukey's or Dunnett's multiple comparison's test (GraphPad Prism). Tukey's multiple comparisons test was used to evaluate the presence of significant differences among all groups, while Dunnett's multiple comparisons test was used to evaluate the presence of significant differences between experimental groups and the control. Means and standard error were computed. Differences were considered statistically significant if p<0.05.

### Results

T. gondii Activates Src in Host Cells that Lack Functional EGFR Signaling and Utilizes Src to Promote Parasite Survival

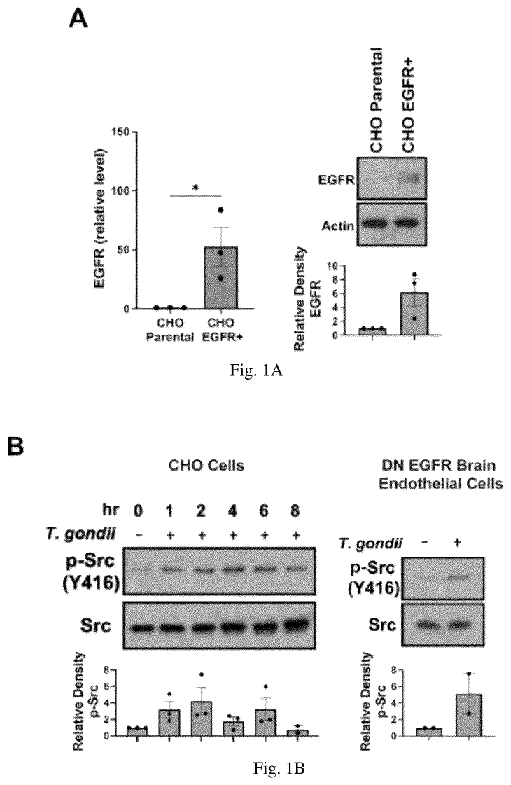

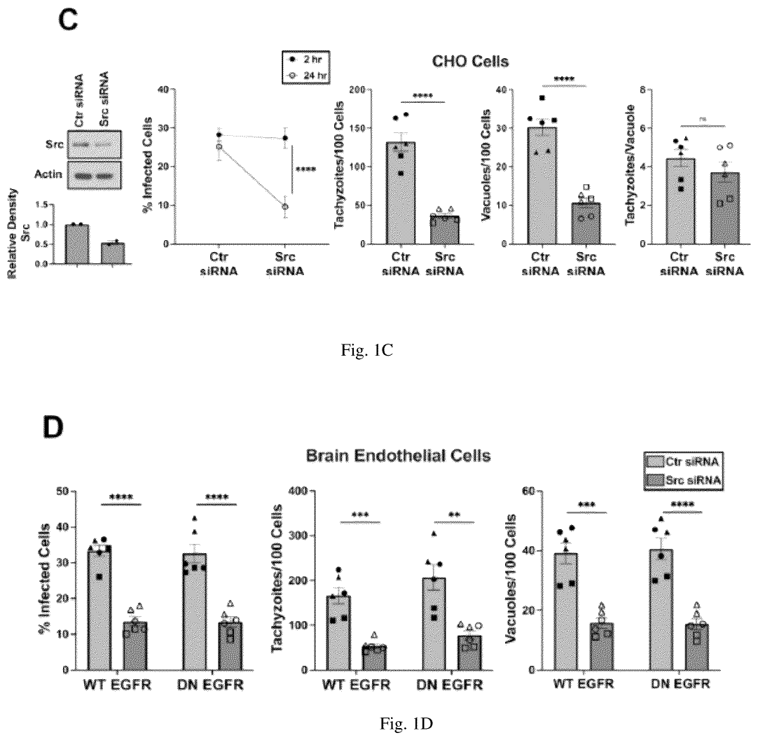

In contrast to EGFR, Src is a ubiquitously expressed signaling molecule. We sought to examine whether T. gondii activates Src in cells that lack EGFR. Parental CHO cells, confirmed to be devoid of EGFR as assessed by real-time quantitative PCR and immunoblot (FIG. 1A), exhibited Src phosphorylation at Tyr416 (marker of Src activation) after infection with T. gondii (FIG. 1B). Similarly, the parasite enhanced Src Tyr416 phosphorylation in brain endothelial cells that express DN EGFR, a mutant previously proven to ablate EGFR signaling (FIG. 1B). Next, we examined whether Src promotes survival of T. gondii in cells that lack EGFR signaling. Knockdown of Src in CHO cells resulted in a decrease in the percentage of infected cells at 24 h vs 2 hr, reduction of the number of vacuoles per 100 cells (both indicative of toxoplasmacidal activity) and a decrease in the number of tachyzoites per 100 cells at 24 hours post-infection (FIG. 1C). Knockdown of Src did not affect the number of tachyzoites per vacuole in the cells that remained infected (FIG. 1C), indicating that deficiency of Src induced parasite killing rather than reducing intracellular replication. Knockdown of Src in primary brain endothelial cells that express DN EGFR or WT EGFR resulted in a reduction in percentage of infected cells, vacuoles per 100 cells, and number of tachyzoites per 100 cells (FIG. 1D).

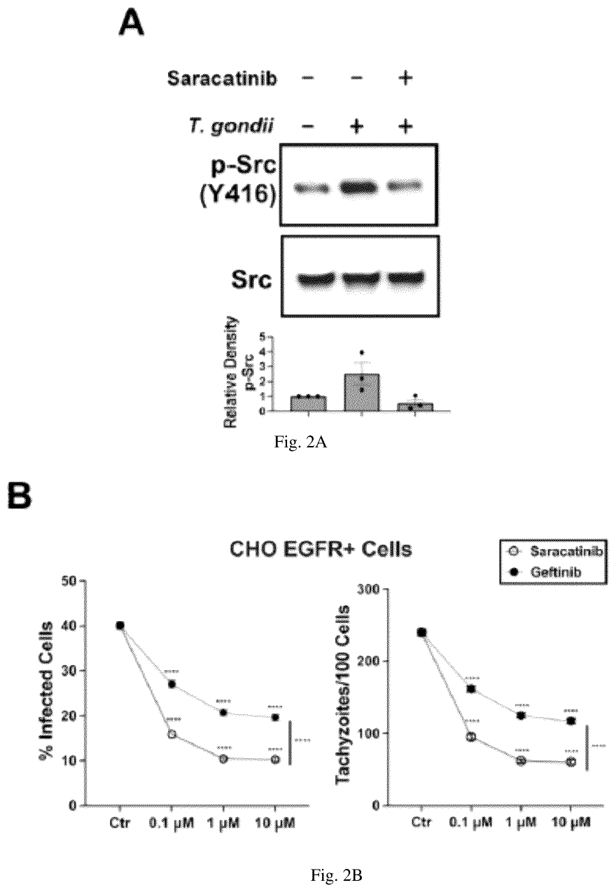

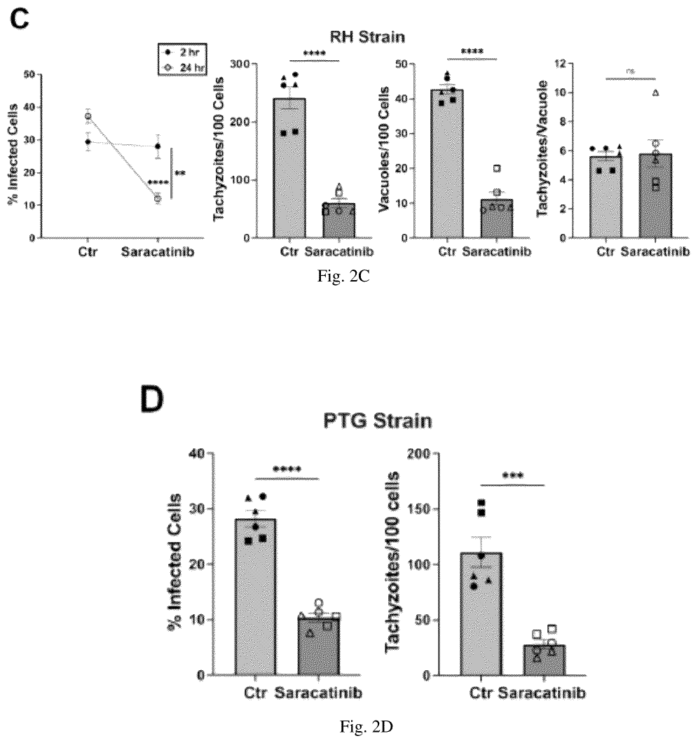

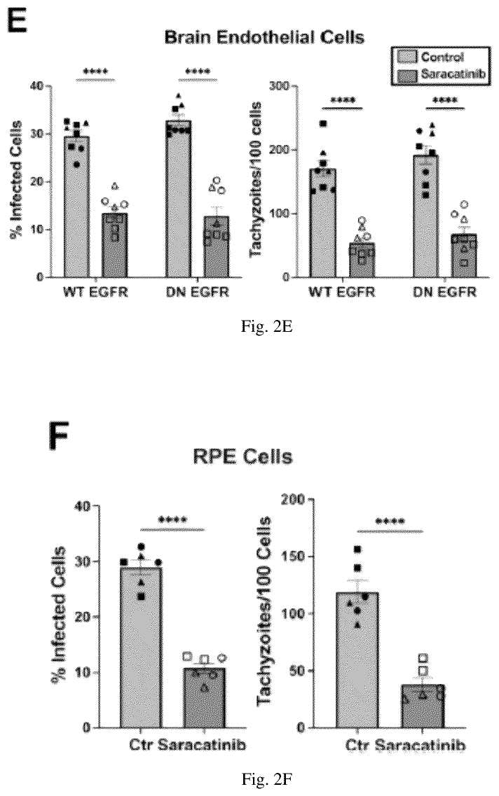

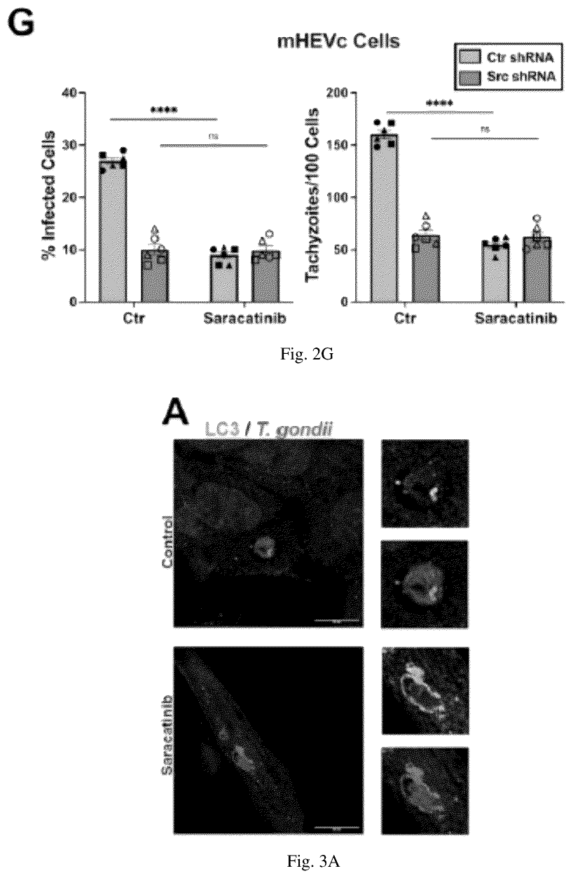

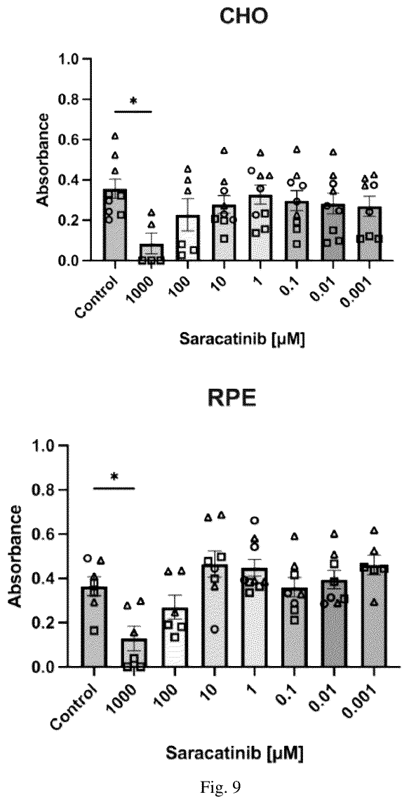

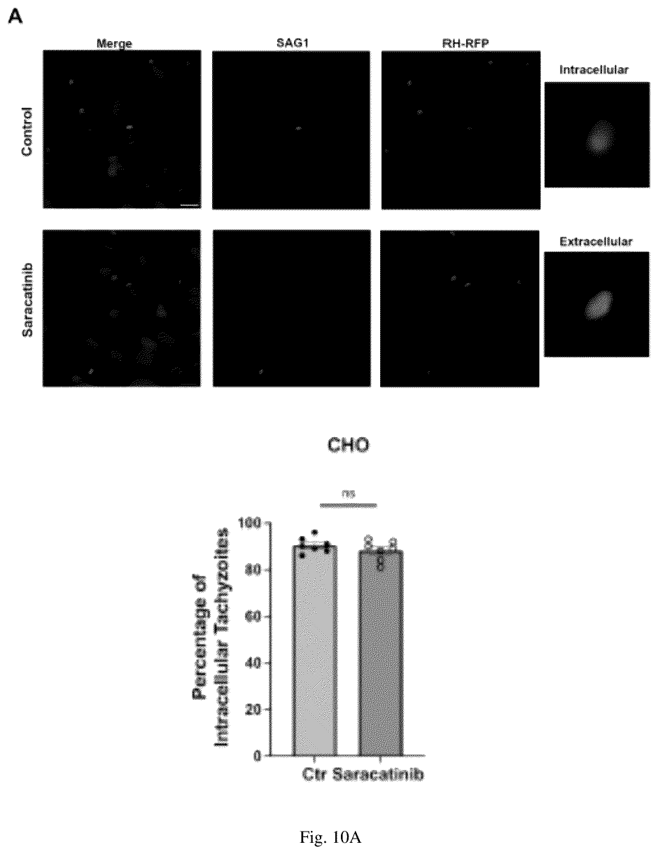

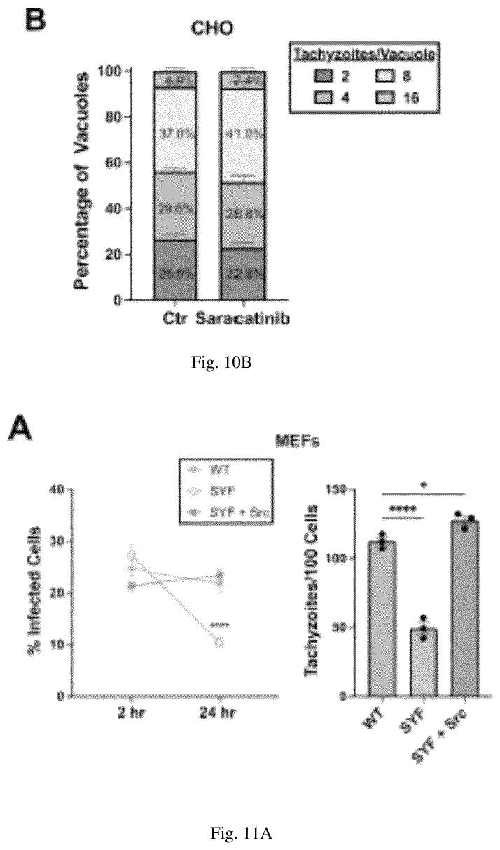

We further examined the effects of inhibition of Src signaling using Saracatinib, a potent and selective Src family kinase inhibitor. Saracatinib markedly impaired T. gondii-induced Tyr416 phosphorylation of Src in CHO cells (FIG. 2A). Moreover, Saracatinib caused a dose-dependent significant decrease in the percentage of infected cells and tachyzoites per 100 cells (FIG. 2B). Those effects were more striking than the effects of Gefitinib, an EGFR family kinase inhibitor, even in CHO cells stably transfected to express EGFR (FIG. 2B). Subsequent studies were performed using Saracatinib at 1 μM, a concentration that was previously reported to be non-toxic in various cell lines, results that were confirmed in CHO and retinal pigment epithelial (RPE) cells (FIG. 9). Saracatinib-induced toxoplasmacidal activity in CHO cells infected with type I or Type II strains of T. gondii (FIGS. 2C and 2D). Saracatinib induced similar effects in brain endothelial cells that expressed WT or non-functional DN EGFR (FIG. 2E), and RPE (cells that express native EGFR; FIG. 2F). Saracatinib did not affect parasite invasion of host cells (FIG. 10A) or parasite replication (FIG. 10B). Importantly, Saracatinib acted on target since no toxoplasmacidal activity was detected if cells were made deficient in Src (FIG. 2G). Moreover, while Saracatinib can also inhibit Fyn, Yes, and c-Abl, studies in cells deficient in Src, Yes and Fyn (SYF) and in cells deficient in only Yes and Fyn (SYF+Src) indicate that deficiency in Src, but not deficiency of Yes plus Fyn, affects survival of T. gondii (FIG. 11A). Similarly, inhibition of c-Abl did not affect parasite survival (FIG. 11B). Taken together, these findings indicate that Src plays a critical role in promoting survival of T. gondii independent of the presence of functional EGFR.

Inhibition of Src in Cells Deficient in EGFR Signaling Induces Autophagy-Mediated Killing of T. gondii.

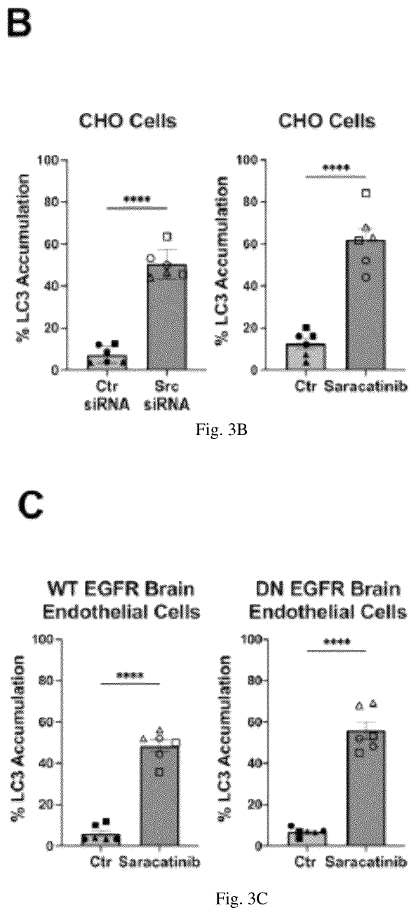

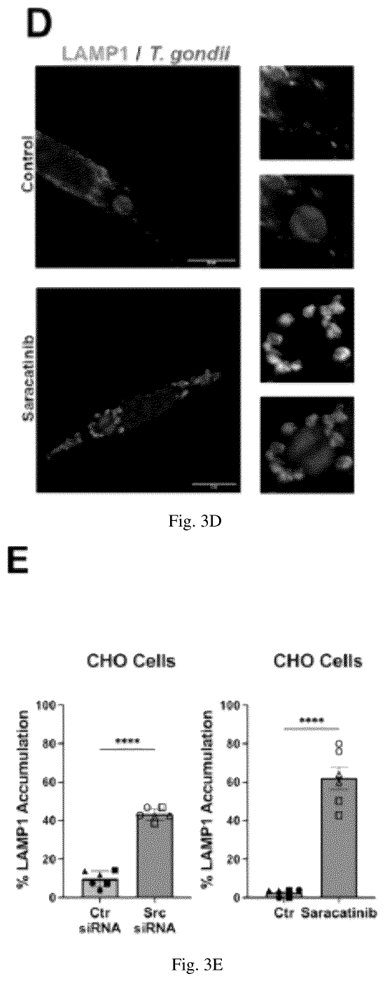

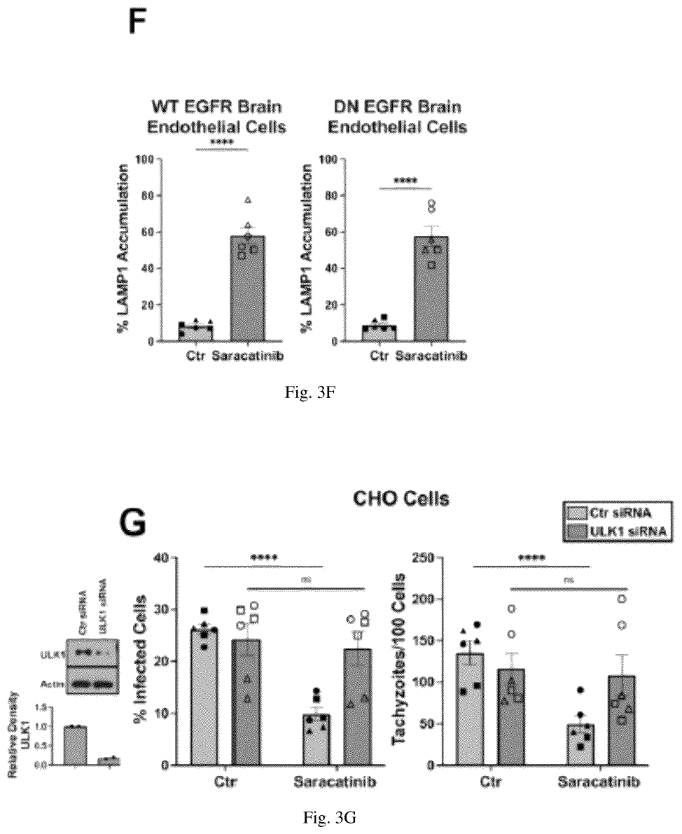

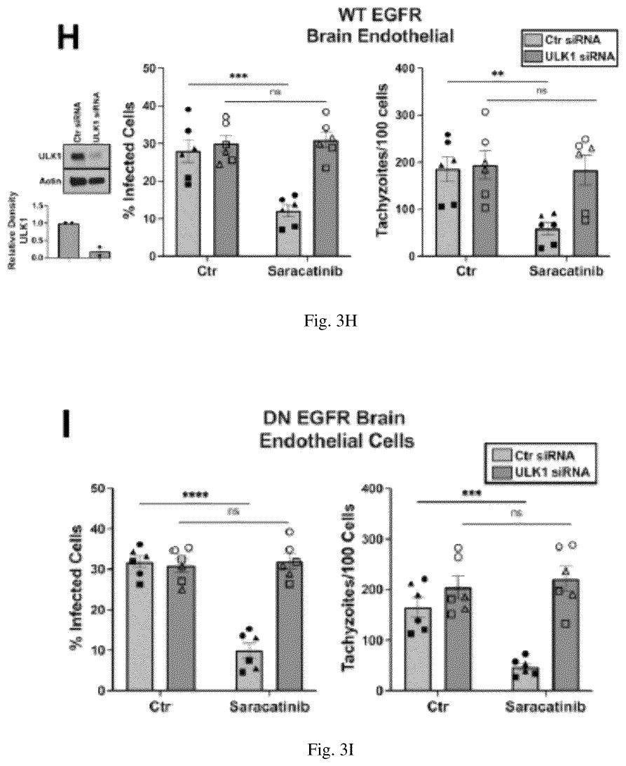

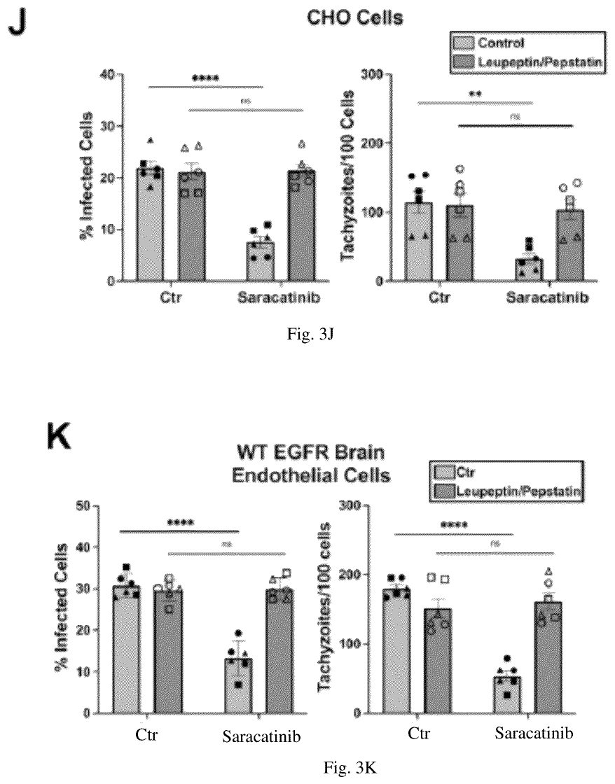

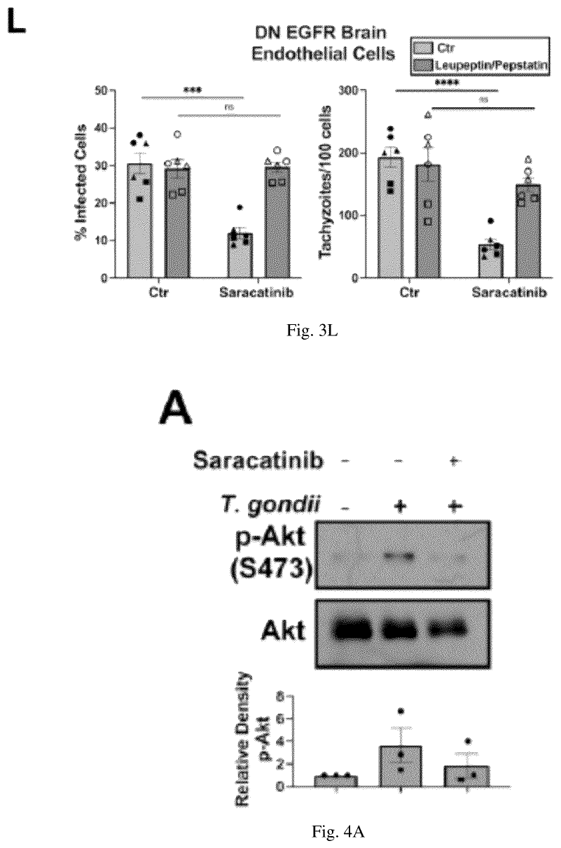

We sought to determine whether Src inhibition triggers autophagy-mediated killing of T. gondii even in cells lacking functional EGFR. Knockdown of Src or treatment with Saracatinib induced accumulation of the autophagosomal marker LC3 around the parasites in CHO cells and brain endothelial cells that express DN EGFR (FIGS. 3A-3C). Similarly, accumulation of the lysosomal marker, LAMP1, was observed around intracellular parasites in cells deficient in Src or treated with Saracatinib (FIGS. 3D-3F). To determine whether killing of T. gondii occurred through autophagy, we examined the effects of knockdown Unc-51-like kinase 1 (ULK1), an upstream inducer of the autophagy cascade required for autophagosome formation.

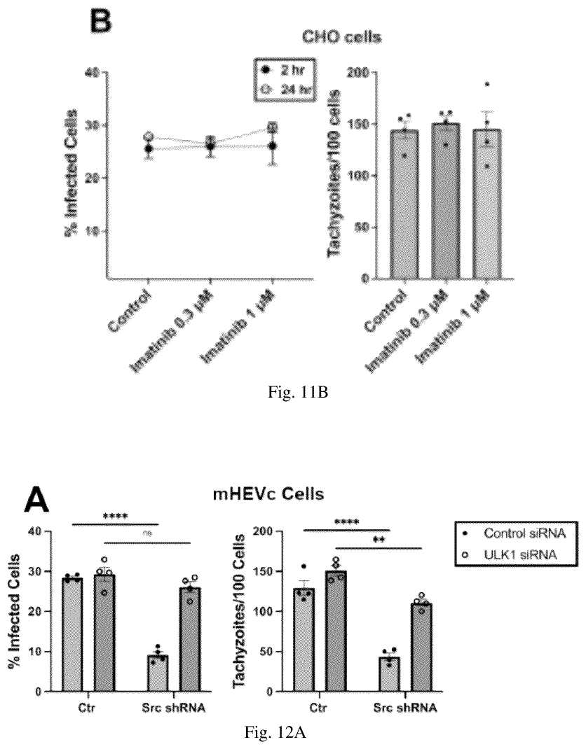

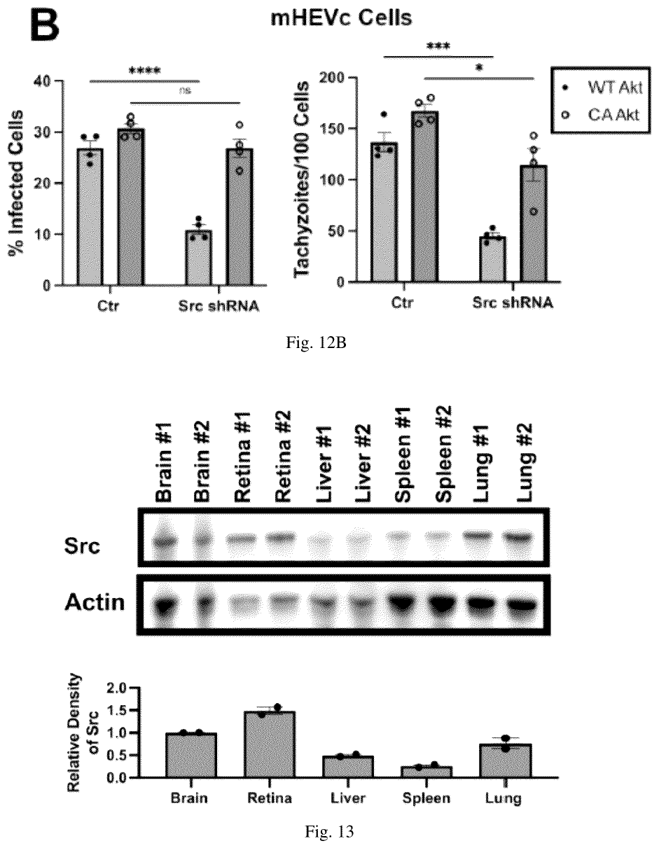

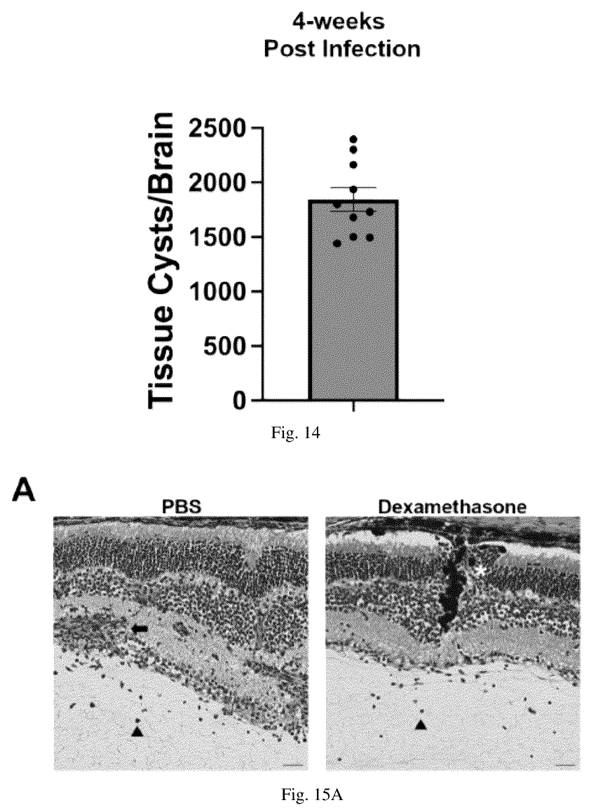

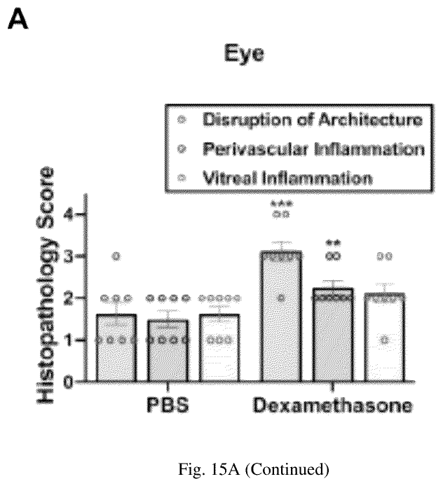

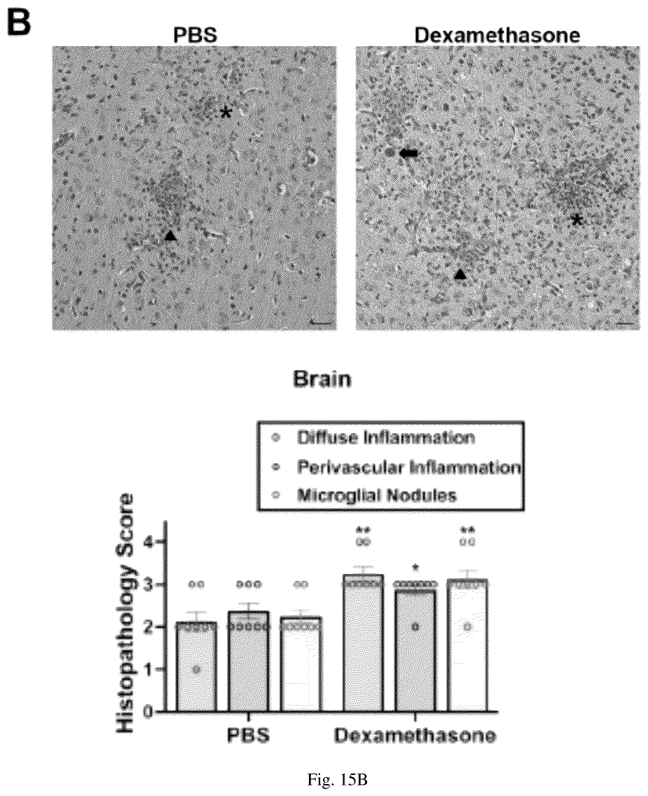

ULK1-deficient cells were unable to exhibit anti-T. gondii activity despite treatment with Saracatinib (FIGS. 3G-3I) or Src knockdown (FIG. 12A). To determine if T. gondii killing was a result of lysosomal degradation, we examined the effects of acid protease inhibitors, Leupeptin and Pepstatin A. Incubation with Leupeptin and Pepstatin A ablated the anti-T. gondii activity induced by Saracatinib (FIG. 3J-3L). Taken together, inhibition of Src induced autophagy-mediated killing of T. gondii even in cells defective in EGFR signaling.

### Src Inhibition in Cells Deficient in EGFR Signaling Induces Killing of T. Gondii by Impairing Akt Activation

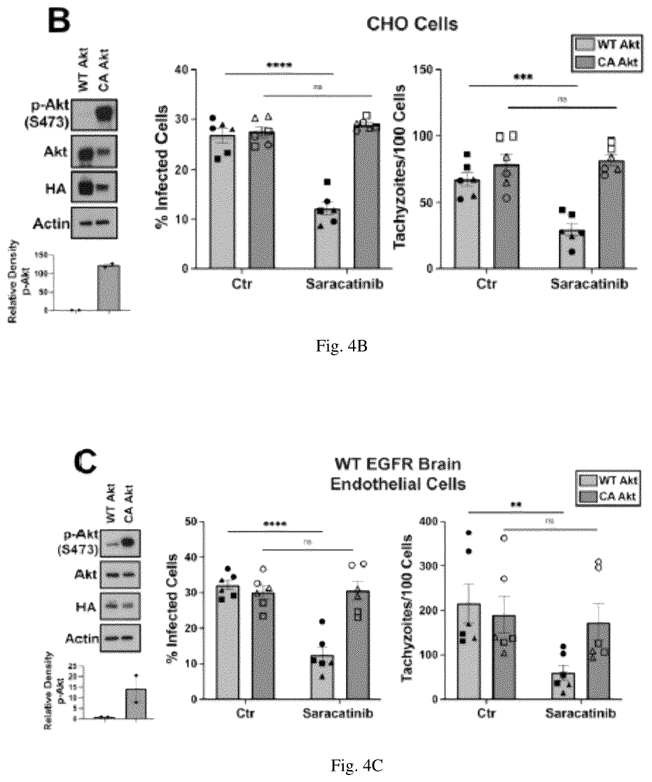

Akt negatively regulates autophagy. We investigated whether Src acts through Akt to prevent T. gondii killing in cells deficient in EGFR signaling. T. gondii infection in CHO cells increased phosphorylation of Akt at the activation site Ser473, an effect that was abrogated by treatment with Saracatinib (FIG. 4A). Next, to determine the role of Akt in regulating the killing of T. gondii induced by Src inhibition, we transfected cells with a plasmid encoding a constitutively active Akt that is directed to membranes by the addition of a Src myristoylation sequence (901 pLNCX myr HA Akt1) or a plasmid encoding a non-myristoylated control as wild-type Akt (903 pLNCX HA Akt1. Transfection with the myristoylated Akt plasmid (referred to as CA Akt) increased the expression of phospho-S473 Akt in comparison to transfection with non-myristoylated Akt plasmid (referred to as WT Akt) (FIG. 4B). Treatment with Saracatinib reduced the percentage of infected cells and the number of tachyzoites per 100 cells in CHO cells transfected with WT Akt (FIG. 4B). In contrast, CA Akt transfected CHO cells treated with Saracatinib showed no reduction in percentage of infection or number of tachyzoites per 100 cells in comparison to untreated cells (FIG. 4B). These results were confirmed in brain endothelial cells (FIG. 4C) as well as in endothelial cells deficient in Src (FIG. 12B). Taken together, these results support that inhibition of Src induces parasite killing by impairing Akt activation.

Inhibition of Src Impairs T. gondii-Induced Akt Activation in a PTEN− Dependent Manner

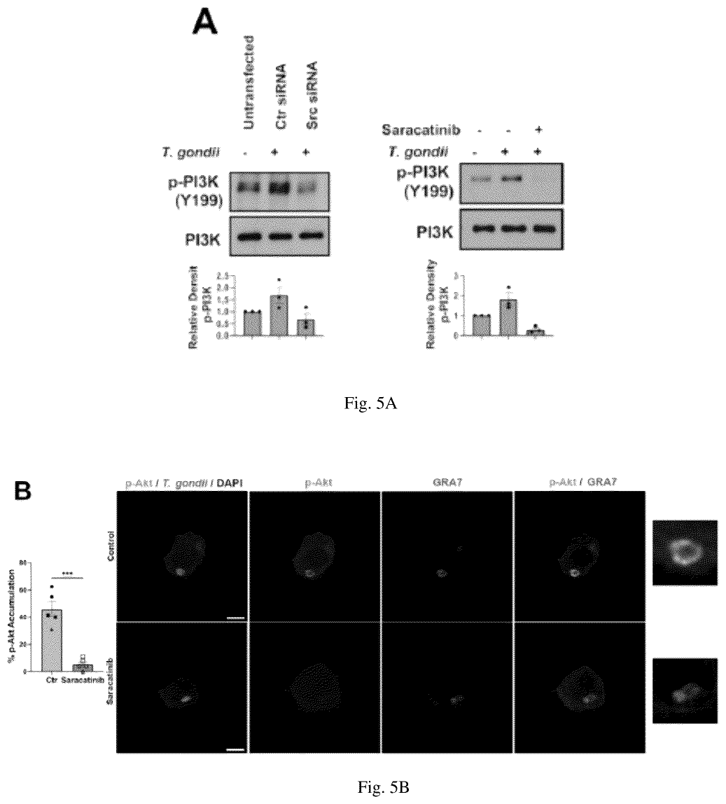

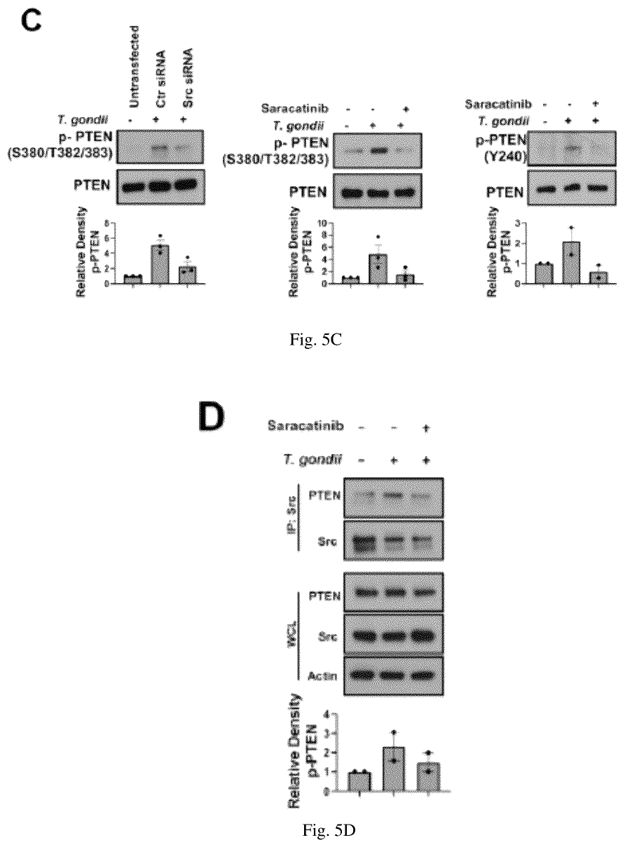

We conducted studies to identify the mechanism through which inhibition of Src regulates T. gondii-induced Akt activation. T. gondii infection in CHO cells increased Y199 phosphorylation of PI3K, a marker of activation of a signaling molecule that increases production of phosphatidylinositols that are key to the activation of Akt (FIG. 5A). Y199 phosphorylation of PI3K was inhibited by Src knockdown or treatment with Saracatinib (FIG. 5A). Infection with T. gondii resulted in accumulation of phospho-Ser473 Akt around the parasite (FIG. 5B). Expression of activated Akt was associated with the PVM since phospho-Ser473 Akt co-localized with the dense granule protein GRA7 (FIG. 5B). Saracatinib inhibited accumulation of phospho-S473 Akt around intracellular parasites (FIG. 5B). Thus, T. gondii promotes Src-dependent expression of activated Akt associated with the PVM.

PTEN acts as a negative regulator of Akt signaling through dephosphorylation of phosphatidylinositols. PTEN binds phospholipids through the C2 domain and functions as a phosphatase. However, phosphorylation at serine and threonine sites (Ser380, Thr382, Thr383) in the regulatory C-terminal tail of PTEN results in interaction between the C-terminal tail and the C2 domain leading to a “closed” inactive conformation. In addition, Src phosphorylates Tyr240 in the C2 domain of PTEN limiting the ability of PTEN to dephosphorylate phosphatidylinositols. Infection with T. gondii resulted in increased phosphorylation of PTEN at Tyr240 that was accompanied by an increase in Ser380 and Thr382/383 phosphorylation, indicating a “closed” conformation with impaired phosphatase activity (FIG. 5C). Src knockdown or Saracatinib treatment in T. gondii-infected cells reduced PTEN phosphorylation at Tyr240 as well as at Ser380 and Thr382/383 (FIG. 5C). Immunoprecipitation studies indicated an increased association of Src and PTEN in T. gondii-infected cells, a phenomenon that was impaired by Saracatinib (FIG. 5D). Membrane association is a feature of biologically active PTEN, an event that is impaired by tyrosine phosphorylation.

Saracatinib resulted in co-expression of PTEN and GRA7 supporting an association of PTEN to the PVM (FIG. 5E). Finally, we examined whether PTEN is required to induce killing of T. gondii in cells treated with Saracatinib. Cells made deficient in PTEN by transfection with siRNA were no longer able to kill T. gondii when treated with Saracatinib. Altogether, T. gondii induces Src-dependent decoration of PVM with activated Akt, an event that is accompanied by PTEN phosphorylation at amino acid residues indicative of a “closed” inactive conformation of PTEN. In contrast, Src inhibition results in dephosphorylation of these amino acids (markers of PTEN activation), translocation of PTEN around the PV and loss of recruitment of activated Akt to the PVM. PTEN is essential for killing T. gondii induced by Src inhibition.

Saracatinib Induces Accumulation of PTEN and Reduces Expression of Activated Akt Around T. gondii in Neural Tissue, as Well as Confers Protection Against Pre-Established Ocular and Cerebral Toxoplasmosis

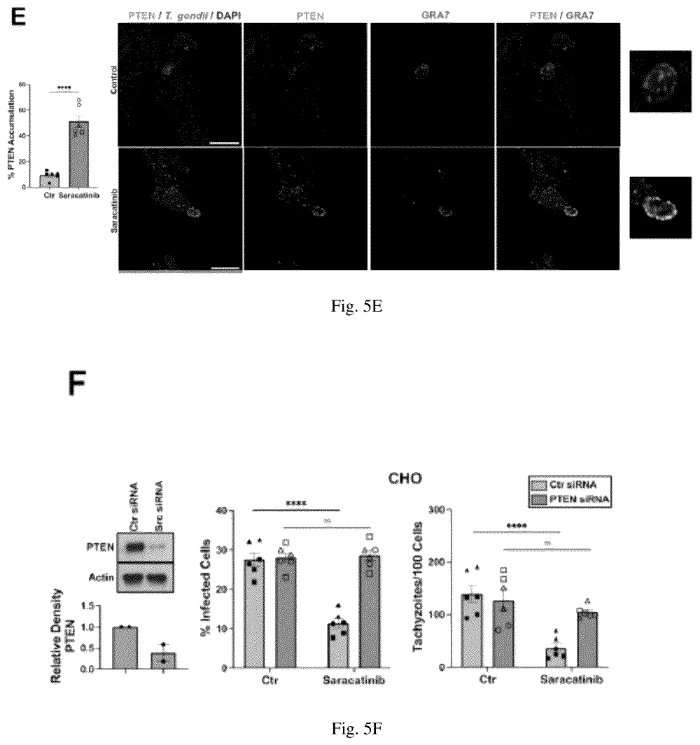

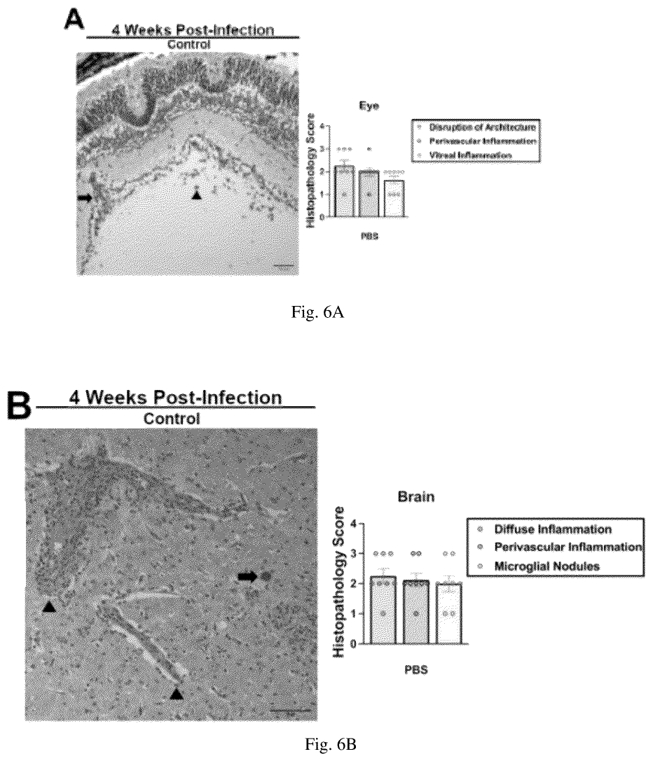

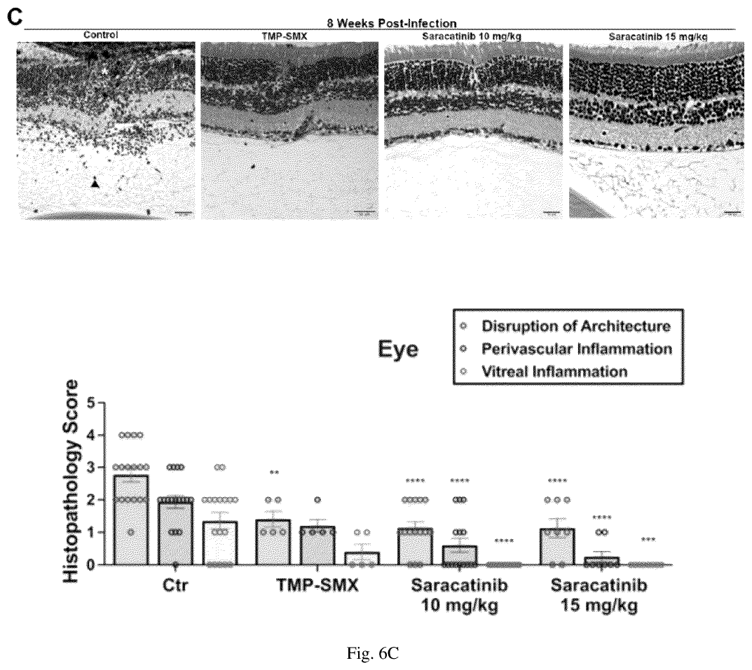

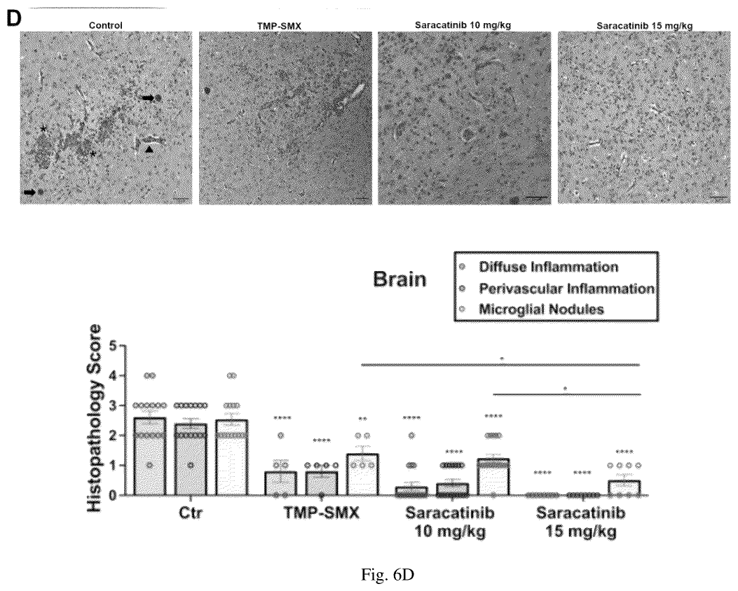

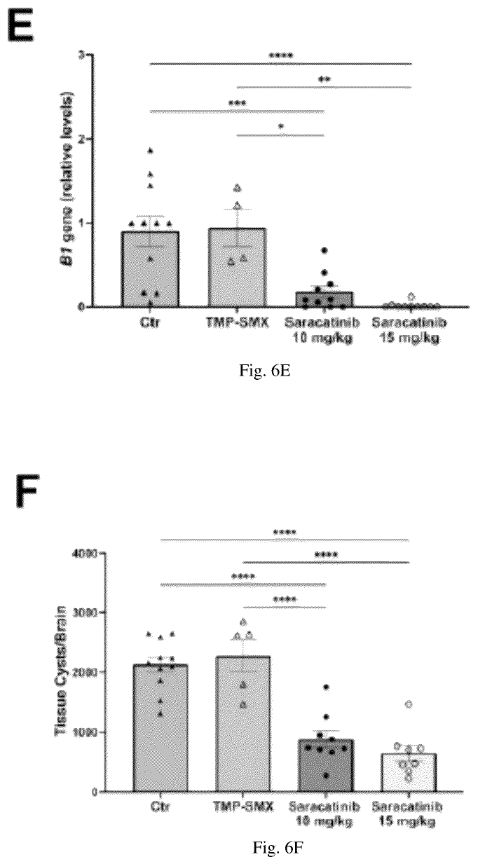

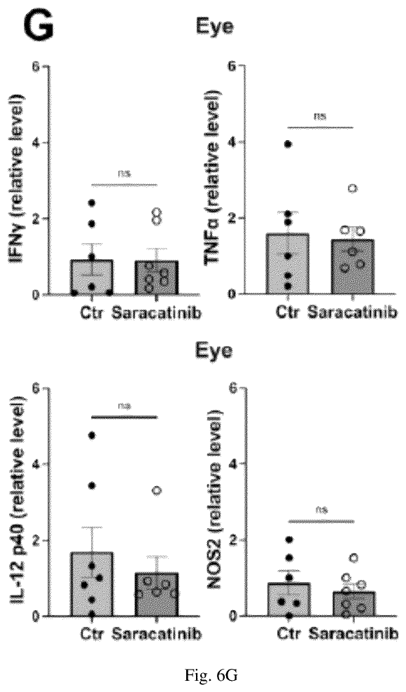

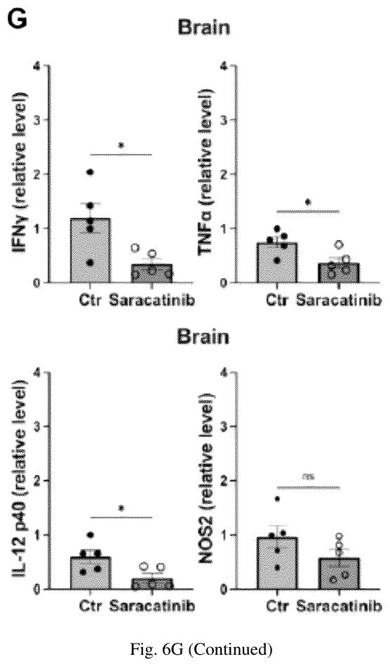

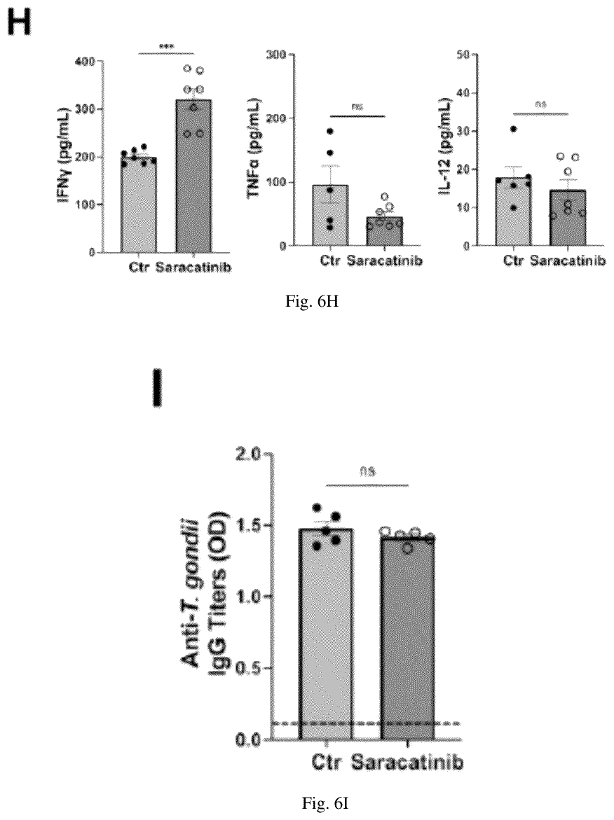

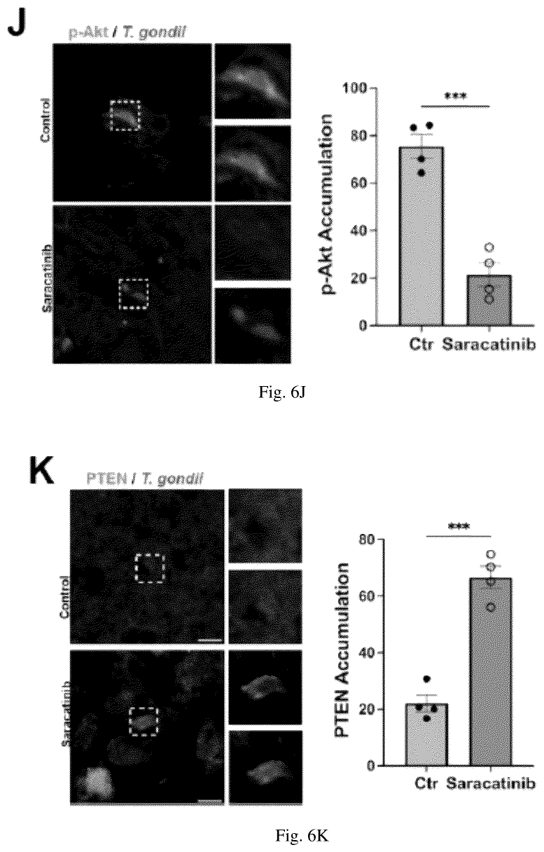

In contrast to EGFR, Src is highly expressed in the brain and retina. Indeed, immunoblot studies revealed high expression of Src in the brain and retina compared to the spleen, liver and lung of B6 mice (FIG. 13). Saracatinib is a well-tolerated drug that penetrates the blood-brain barrier. Saracatinib is approved for the treatment of idiopathic pulmonary fibrosis and has been used in various clinical trials including in patients with Alzheimer's and Parkinson's disease. B6 mice infected with tissue cysts of ME49 T. gondii for four weeks showed histopathology in the eye and brain indicative of ocular and cerebral toxoplasmosis (FIGS. 6A and 6B). In addition, infected mice had a high tissue cyst burden at this time post-infection. B6 mice infected for four weeks (with pre-established ocular and cerebral toxoplasmosis) were then treated with Saracatinib for four weeks (10 mg kg⁻¹ or 15 mg kg⁻¹ twice per day, doses predicted to achieve brain concentrations˜0.2 μM), or vehicle. Mice treated with Saracatinib had a profound reduction in parasite load in the eye and brain as well as a marked reduction in histopathology in these organs (FIG. 6C-6F). No significant ocular inflammation was detected in 75% of the mice treated with the 15 mg kg⁻¹ dose (FIGS. 6C and 6D). No significant perivascular and diffuse inflammation were detected in the brains of those mice (FIGS. 6C and 6D). In parallel experiments, mice treated with a high-dose of Trimethoprim-Sulfamethoxazole, an antibiotic regimen reported to be equally effective, but better tolerated than Pyrimethamine plus Sulfadiazine in patients with cerebral toxoplasmosis, exhibited a less pronounced reduction in histopathology in the eye and brain compared to Saracatinib (FIGS. 6C and 6D). Protection against ocular and cerebral toxoplasmosis in Saracatinib-treated mice was not the result of increased local expression of IFN-γ, TNF-α, IL-12 p40, and NOS2, mediators of resistance against ocular and cerebral toxoplasmosis (FIG. 6G). Saracatinib did not affect serum levels of TNF-α and IL-12 p40 and caused a modest increase in serum IFN-γ levels (FIG. 6H) consistent with the report that low doses of Saracatinib can enhance IFN-γ production. Anti-T. gondii IgG levels were unchanged in mice treated with Saracatinib (FIG. 6I).

Next, we examined the expression of activated Akt and PTEN in vivo. Tachyzoites were present in the brains of mice with cerebral toxoplasmosis and immunofluorescence in brain sections from infected mice that received vehicle demonstrated accumulation of phospho-S473 Akt around tachyzoites (FIG. 6J). Saracatinib markedly reduced phospho-S473 Akt accumulation. Moreover, Saracatinib induced recruitment of PTEN around tachyzoites (FIG. 6K).

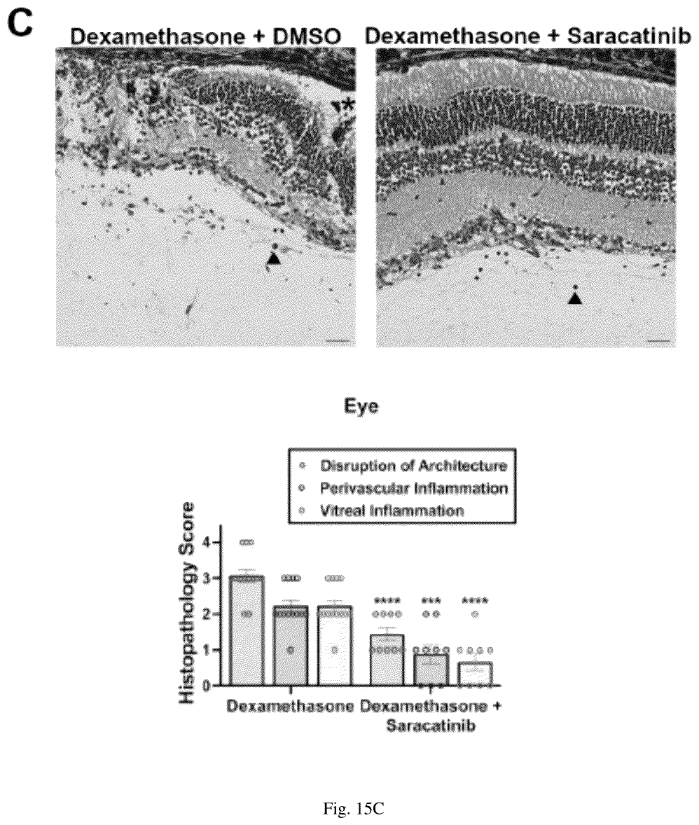

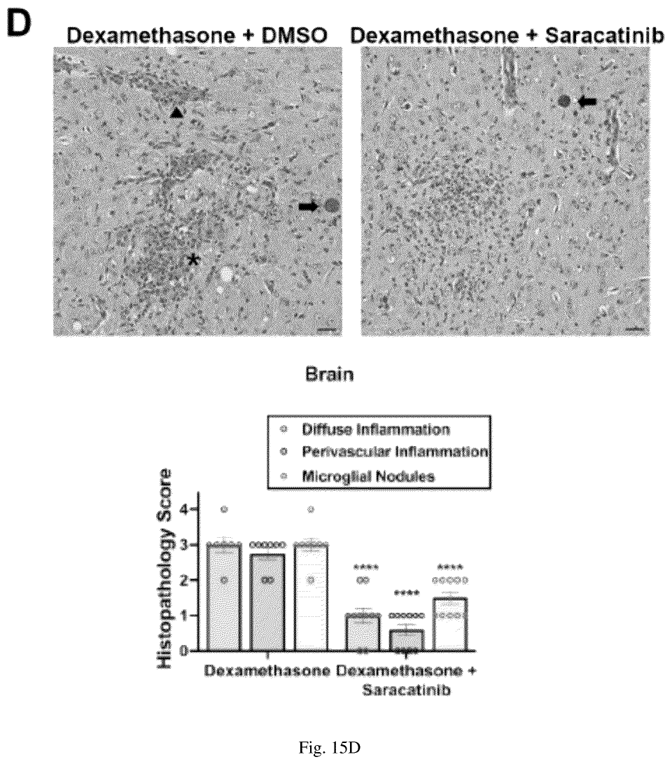

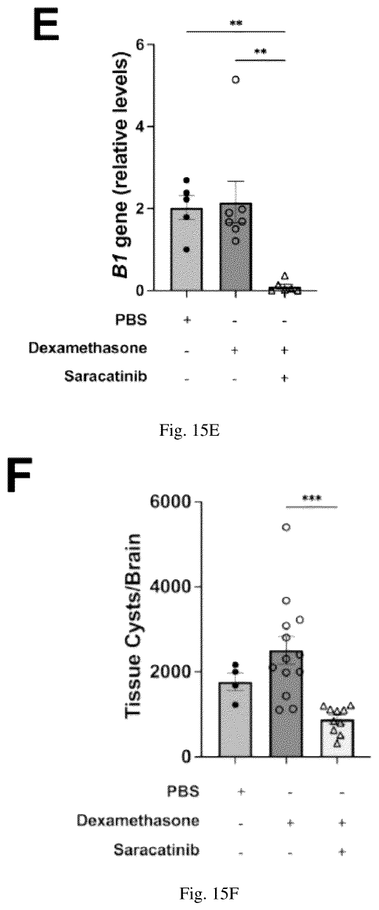

To further explore the effects of Saracatinib, we used chronically infected mice where neural toxoplasmosis had been exacerbated by an immunosuppressive agent. B6 mice infected with ME49 T. gondii tissue cysts for 3 weeks were given Dexamethasone (0.1 mg kg-1, once per day) for 2 weeks. Examination of brains and eyes revealed that ocular and cerebral toxoplasmosis were exacerbated by Dexamethasone (FIGS. 15A-B). After 2 weeks of Dexamethasone administration, infected mice were treated with either vehicle or Saracatinib (10 mg kg-1 twice per day) for 2 weeks while Dexamethasone administration was continued. Saracatinib significantly reduced histopathology and parasite load in the eye and brain compared to vehicle-treated mice (FIG. 15C-F). Thus, Saracatinib effectively controlled ocular and cerebral toxoplasmosis in chronically infected mice, even if the animals received an immunosuppressant.

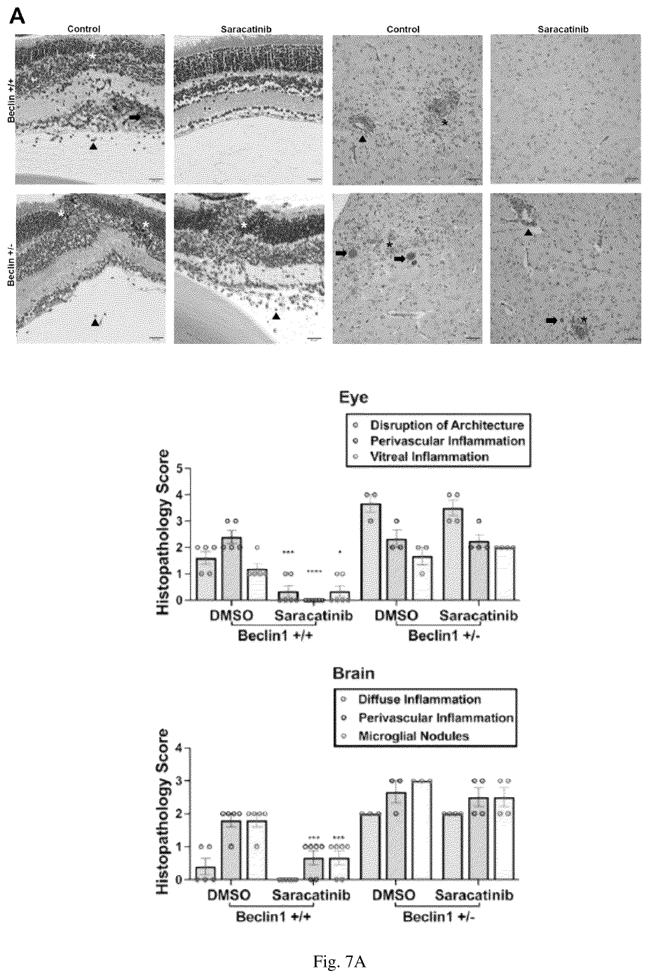

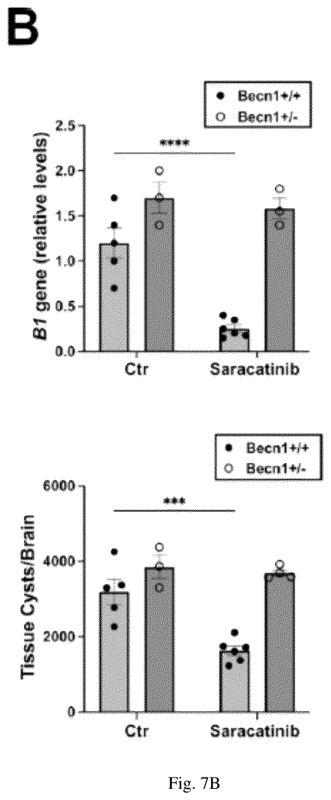

Finally, we determined if the protective effect of Saracatinib was dependent on the autophagy protein Beclin 1. Becn 1^{+/+} and Becn 1^{+/-} mice that had been infected with ME49 tissue cysts for four weeks to develop pre-established disease were then treated with Saracatinib for 2 weeks. Saracatinib-treated Becn 1^{+/+} mice demonstrated improvement in ocular and cerebral histopathology as well as parasite load (FIG. 7A). In contrast, autophagy-deficient Becn 1^{+/-} mice did not respond to Saracatinib administration and remained with severe histopathology and elevated parasite loads (FIGS. 7A and 7B). Taken together, Saracatinib triggered accumulation of PTEN around tachyzoites in neural tissue reducing expression of phospho-S473 Akt, triggered marked reduction in parasite load and induced protection against ocular and cerebral toxoplasmosis that were dependent on Beclin 1.

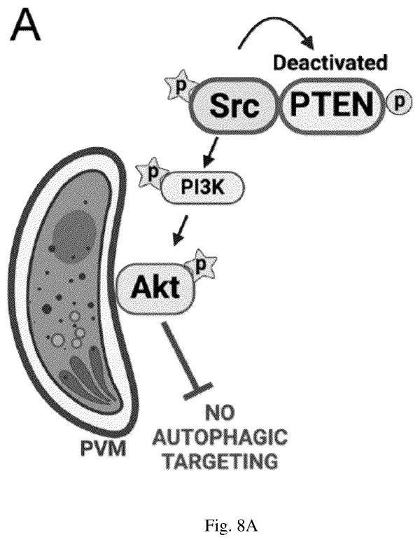

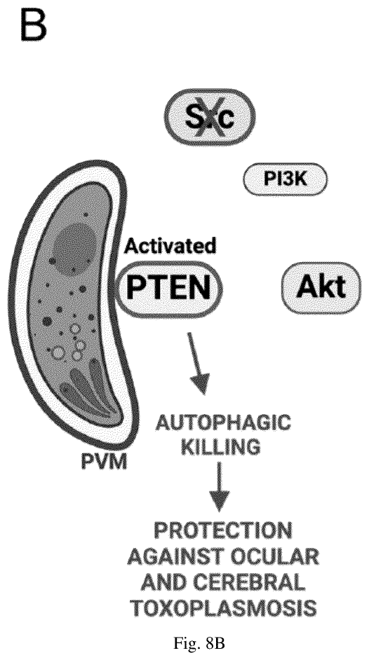

Avoidance of autophagy is key to the survival of T. gondii since this protozoan is unable to withstand the lysosomal environment. We report that the ubiquitously expressed signaling molecule, Src, is essential for preventing autophagic targeting of T. gondii, an effect that does not require the presence of EGFR, the host cell molecule previously reported to protect the parasite against autophagy (FIG. 8). Knockdown of Src or pharmacologic inhibition of Src using Saracatinib resulted in entrapment of T. gondii by an LC3⁺ structure, vacuole-lysosomal fusion and killing of the parasite dependent on ULK1 and lysosomal enzymes. Src promoted activation of Akt, a central negative regulator of autophagy. Parasite killing in cells deficient in Src signaling was ablated by expression of CA-Akt, indicating that Src promoted parasite survival through Akt activation. Src regulated the activity of Akt via PTEN. Src associated with PTEN in T. gondii-infected cells, an effect that was dependent on Src activation (FIG. 8). Src promoted an inactive conformation of PTEN and therefore, PTEN was not detected around the PV. However, PTEN acquired an active conformation and migrated around the PV in cells treated with Saracatinib. This was accompanied by marked reduction in the expression of activated Akt in the PVM and autophagic killing of T. gondii. Treatment of mice with ocular and cerebral toxoplasmosis with Saracatinib led to replacement of activated Akt for PTEN around the tachyzoites, marked reduction in parasite load and histopathology that were dependent on the autophagy protein Beclin 1. In summary, these results uncovered the importance of the Src-PTEN-Akt pathway as a central regulator of T. gondii survival and support the key role of this pathway in neural toxoplasmosis.

Our studies revealed that activated Akt surrounded intracellular tachyzoites. In this regard, increasing evidence supports that Akt is not only functional at the level of the plasma membrane but may also act in intracellular signaling hubs whereby cytoplasmic Akt would be recruited to compartments enriched for phosphatidylinositols leading to allosteric activation of membrane-associated Akt. Activated Akt within T. gondii-infected cells co-localized with GRA7 supporting that it is associated with the PVM. This finding together with the evidence that activated Akt promotes T. gondii survival supports that this molecule acts at the level of the PVM to protect the parasite. Such spatial compartmentalization of Akt activation to the PVM would result in a specific effect targeted against this compartment. Of note, Akt in the lysosomal membrane can regulate chaperone-mediated autophagy.

PTEN is a major negative regulator of Akt signaling by acting as a phosphatase against phosphatidylinositols. Our studies revealed that Src functions through PTEN to regulate activation of Akt in T. gondii-infected cells. Src associated with PTEN in T. gondii-infected cells. This was accompanied by phosphorylation of PTEN in Y240, a residue reported to be directly phosphorylated by Src, and by maintenance of S380, T382 and T383 phosphorylation, a hallmark of the inactive conformation of PTEN. However, genetic, or pharmacologic inhibition of Src resulted in dephosphorylation Y240, S380, T382 and T383 of PTEN, markers of activated PTEN. Indeed, PTEN was recruited around the PV, and this was accompanied by marked reduction in the accumulation activated Akt. Moreover, PTEN was required for killing of T. gondii upon inhibition of Src signaling. In addition to promoting the inactive state of PTEN, Src promotes activation of PI3K, a molecule that stimulates phosphatidylinositol expression. PI3K has been reported to activate Akt in T. gondii-infected cells, an event that occurs in both an EGFR-dependent and independent manner. Taken together, there are at least two levels at which Src controls the Akt pathway in T. gondii-infected cells: deactivation of PTEN and activation of PI3K.

T. gondii activates Src during host cell invasion by triggering integrin-dependent Focal Adhesion Kinase activation. The parasite maintains Src activation during its intracellular state through parasite-induced PKCα/β signaling. While this work uncovered that Src is central for avoidance of autophagic targeting of T. gondii and parasite survival in cells deficient in EGFR, Src is also a component of the EGFR-dependent strategy utilized by the parasite to avoid killing by autophagy. Src activates EGFR in cells that express this receptor which in turn promotes Akt activation. Here, we show that Src can directly regulate Akt activation in the absence of EGFR. It is likely that both mechanisms of regulation of Akt activity are operative in cells that express EGFR.

Saracatinib penetrates the blood-brain barrier and is well tolerated in animals and humans, including elderly individuals. Our studies indicate that the effects of Saracatinib in T. gondii infection are mediated by inhibition of Src since the anti-T. gondii activity of Saracatinib was dependent on Src (not present in Src-deficient cells), and yes, Fyn as well as c-Abl do not play an appreciable role in regulating survival of T. gondii. Saracatinib did not exhibit appreciable direct anti-parasitic activity since the replication of T. gondii was unaffected in cells that remained infected following treatment. Moreover, Saracatinib replicated the effect of Src knockdown further supporting that this drug acts as a Src inhibitor in models of T. gondii infection.

Our in vivo studies indicated that the interplay between Src, PTEN and Akt takes place around tachyzoites in neural tissue. We chose to examine the in vivo relevance of this cascade by testing the effects of a Src kinase inhibitor due to the high level of Src expression in neural tissue (approximately 8-10 times than other cells), the ability of Saracatinib to induce potent inhibition of Src signaling and penetrate the blood brain barrier. The fact that Saracatinib induced recruitment of PTEN around tachyzoites, diminished expression of activated Akt and markedly reduced the parasite load in the brain and eye in a Beclin 1-dependent manner supports that the Src-PTEN-Akt dependent regulation of autophagic control of T. gondii is operative in vivo in neural tissue.

Whereas Saracatinib can control an intracellular pathogen in vivo, it should be considered that Src family kinases have been implicated as signaling molecules downstream of receptors in immune cells. However, their role is considered to be that of modulators of responses in these cells rather than “on-off switches”. This is because these kinases can trigger stimulatory or inhibitory pathways and they can exhibit redundant effects in immune cells. Moreover, in vitro and in vivo studies revealed that administration of low doses of Saracatinib (similar to those used in our work) to previously primed T cells resulted in increased T cell expansion and IFN-γ production following antigenic stimulation. Interestingly, Saracatinib did not inhibit the activity of Lek and Fyn in T cells (Src family kinases involved in TCR signaling) whereas the Src family kinase inhibitor Dasatinib reduced Lek and Fyn kinase activity in T cells and impaired IFN-γ production. Indeed, our studies revealed that Saracatinib caused a modest increase in serum levels of IFN-γ in T. gondii-infected mice and did not affect serum levels of TNF-α and IL-12 p40. The reduction on IFN-γ, TNF-α, IL-12 p40 and NOS2 mRNA levels in the brain and eye are consistent with the profound decrease in parasite load in these organs.

While we used Saracatinib as a pharmacological tool to probe the role of a host kinase in cerebral and ocular toxoplasmosis, the striking effects of this drug in reducing parasite load and histopathology support that Saracatinib can have therapeutic applications in the treatment of ocular and cerebral toxoplasmosis. Finding that Saracatinib and anti-T. gondii antibiotics cooperate to control the parasite can result in novel and improved therapeutic regimens based on the administration of Saracatinib at doses even lower than those used in this study plus administration of low doses of antibiotics with the goal to achieve optimal control of toxoplasmosis while minimizing side-effects.

In summary, we identified that Src acts as a major determinant of T. gondii survival by maintaining PTEN in a deactivated state and thus, promoting an intracellular signaling hub consisting of activated Akt associated with the PVM that protects the parasite against autophagic killing. Inhibition of Src results in an activated state of PTEN, recruitment of PTEN around the PV and reduction in the expression of activated Akt. As a result, T. gondii is no longer protected against autophagic killing. These findings are important because these cascades of events are not restricted by the presence of EGFR (in contrast to strategies of parasite survival identified previously) and are operative in neural tissue.

### Example 2

### Effect of Combination of Suboptimal Concentrations of Saracatinib and Antibiotics

While Saracatinib at 1 μM kills T. gondii within infected host cells, toxoplasmacidal activity is significantly reduced at 0.3 μM. We treated retinal pigment epithelial cells infected with RH T. gondii with suboptimal concentrations of Saracatinib (0.3 μM) with or without suboptimal concentrations of the anti-T. gondii antibiotic Pyrimethamine (0.1 μM). The combination of Saracatinib plus Pyrimethamine exhibited markedly increased toxoplasmacidal activity. Similar results are observed with the combination of Saracatinib and suboptimal concentration of the antibiotic Trimethoprim-sulfamethoxazole (0.67/3.3 μg/ml).

### Example 3

### Effect of Combination of Suboptimal Concentrations of Saracatinib and Gefitinib

Retinal pigment epithelial cells infected with RH T. gondii were treated with suboptimal concentrations of Saracatinib (0.3 μM) with or without suboptimal concentrations of Gefitinib (1 μM). The combination of Saracatinib plus Gefitinib exhibited markedly increased toxoplasmacidal activity.

### Example 4

Effect of Combination of Suboptimal Dose of Saracatinib and Antibiotics in Mice with Ocular and Cerebral Toxoplasmosis

C57BL/6 mice that had been infected with tissue cysts of ME49 T. gondii for 4 weeks (at a time when animals have developed ocular and cerebral toxoplasmosis), were treated with suboptimal dose of Saracatinib (2.5 mg/Kg once a day) with or without suboptimal dose of Pyrimethamine (4.2 mg/Kg once a day) for 14 days. Whereas neither the low dose Saracatinib nor the low dose Pyrimethamine decreased the load of tissue cysts, the drug combination resulted in a significant reduction (50%) in tissue cysts in the brain after the short course of treatment. Similarly, a marked reduction in parasite load (T. gondii B1 gene) in the eye was observed in mice treated with the drug combination.

### Example 5

### Effect of Saracatinib on Parasite Cysts

None of the antibiotics used against T. gondii exhibit activity against parasite cysts. We infected CHO cells with EGS T. gondii, a strain of the parasite that spontaneously form cysts in vitro. Treatment of CHO cells containing cysts with Saracatinib (1 μM) for 4 days caused a significant reduction (˜60% reduction) in the number of cysts. Many of the remaining cysts had bradyzoites with altered morphology. The reduction in cysts appeared to be more profound (˜78%) in cells treated with Saracatinib plus Gefitinib (1 μM)

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, we claim:

1. A method of inducing killing and/or degradation of an intracellular pathogen in a subject in need thereof, the method comprising:
administering to the subject an amount of an Src family kinase inhibitor effective to inhibit intracellular pathogen activation of Src and AKT in the subject, wherein the intracellular pathogen activates Src and AKT to avoid killing and/or degradation of the pathogen in the subject.

2. The method of claim 1, wherein the intracellular pathogen comprises Toxoplasma gondii.

3. The method of claim 1, wherein the subject has ocular and/or cerebral toxoplasmosis.

4. The method of claim 1, wherein the Src family kinase inhibitor is administered at a therapeutic dose or subtherapeutic dose.

5. The method of claim 1, wherein the Src family kinase inhibitor comprises at least one of N-(2-chloro-6-methylphenyl)-2-[[6-[4-(2-hydroxyethyl)-1-piperazinyl]-2-methyl-4-pyrimidinyl]amino]-5-thiazole carboxamide monohydrate (Dasatinib), N-(5-chloro-1,3-benzodioxol-4-yl)-7-(2-(4-methylpiperazin-1-yl)ethoxy)-5-(tetrahydro-2H-pyran-4-yloxy)quinazolin-4-amine (saracatinib), 4-[(2,4-dichloro-5-methoxyphenyl)amino]-6-methoxy-7-[3-(4-methylpiperazin-1-yl)propoxy]quinoline-3-carbonitrile (bosutinib), (4-Amino-5-(4-methylphenyl)-7-(t-butyl)pyrazolo[3,4-d]-pyrimidine), PP2 (4-chlorophenyl)-7-(t-butyl)pyrazolo[3,4-d]-pyrimidine) (PP1), 1-tert-butyl-3-(4-chlorophenyl)pyrazolo[3,4-d]pyrimidin-4-amine (PP2), 6-(2,6-dichlorophenyl)-2-{[3-(hydroxymethyl)phenyl]amino}-8-methyl-7H,8H-pyrido[2,3-d]pyrimidin-7-one (PD1663266), (E)-N-[4-[3-chloro-4-(pyridin-2-ylmethoxy)anilino]-3-cyano-7-ethoxyquinolin-6-yl]-4-(dimethylamino)but-2-enamide (neratinib), 3-(2-imidazo[1,2-b]pyridazin-3-ylethynyl)-4-methyl-N-[4-[(4-methylpiperazin-1-yl)methyl]-3-(trifluoromethyl)phenyl]benzamide (ponatinib), (E)-N-[4-(3-chloro-4-fluoroanilino)-3-cyano-7-ethoxyquinolin-6-yl]-4-(dimethylamino)but-2-enamide (pelitinib), N-benzyl-2-[5-[4-(2-morpholin-4-ylethoxy)phenyl]pyridin-2-yl]acetamide (Tirbanibulin), 4-methyl-3-[(2-methyl-6-pyridin-3-ylpyrazolo[3,4-d]pyrimidin-4-yl)amino]-N-[3-(trifluoromethyl)phenyl]benzamide (NVP-BHG712), (2S,3S)-2,3-dihydroxybutanedioic acid; 6-(4-methylpiperazin-1-yl)-N-(5-methyl-1H-pyrazol-3-yl)-2-[(E)-2-phenylethenyl]pyrimidin-4-amine (ENMD-2076), 4-[4-[(5-tert-butyl-2-quinolin-6-ylpyrazol-3-yl)carbamoylamino]-3-fluorophenoxy]-N-methylpyridine-2-carboxamide (Rebastinib), analogues thereof, or any combination thereof.

6. The method of claim 1, wherein the Src family kinase inhibitor is saracatinib (AZD0530) or an analogue thereof.

7. The method of claim 1, wherein the Src family kinase inhibitor is administered to the subject at a dose of less than about 150 mg per day.

8. The method of claim 1, wherein the Src family kinase inhibitor is administered at an amount effective to provide a plasma drug level less than about 250 ng/ml.

9. The method of claim 1, further comprising administering an antimicrobial agent and/or EGFR inhibitor in combination with the Src family kinase inhibitor.

10. The method of claim 9, wherein the antimicrobial agent is an antibiotic agent, antiprotozoal agent, and/or antiparasitic agent.

11. The method of claim 9, wherein the antimicrobial agent and/or EGFR inhibitor is administered at a subtherapeutic dose with a subtherapeutic dose of the Src family kinase inhibitor, wherein the killing and/or degradation effect of the Src inhibitor on the pathogen is enhanced as compared to the effect of the Src inhibitor administered without the antimicrobial agent and/or EGFR inhibitor.

12. The method of claim 9, wherein the antimicrobial agent comprises at least one of amoxicillin, atovaquone, diaminopyrimidines, especially amodiaquine, amphotericin, proguanil, chloroquine, clindamycin, eflornithine, furazolidone, a fluoroquinolone, a third generation cephalosporin, hydroxychloroquine, mefloquine, melarsoprol, metronidazole, minocycline, nifursemizone, nitazoxanide, ornidazole, paromycin sulfate, pentamidine, pyrimethamine, quinapyramine, ronidazole, tinidazole, spriramycin, sulfadiazine, sulfamethoxazole, trimethoprim, analogues thereof, or combinations thereof.

13. A method of treating ocular and/or cerebral toxoplasmosis in a subject in need thereof, the method comprising:
administering to the subject an amount of an Src family kinase inhibitor effective to inhibit Toxoplasma gondii activation of Src and AKT in the subject.

14. The method of claim 13, wherein the Src family kinase inhibitor is administered at a therapeutic dose or subtherapeutic dose.

15. The method of claim 13, wherein the Src family kinase inhibitor comprises at least one of N-(2-chloro-6-methylphenyl)-2-[[6-[4-(2-hydroxyethyl)-1-piperazinyl]-2-methyl-4-pyrimidinyl]amino]-5-thiazole carboxamide monohydrate (Dasatinib), N-(5-chloro-1,3-benzodioxol-4-yl)-7-(2-(4-methylpiperazin-1-yl)ethoxy)-5-(tetrahydro-2H-pyran-4-yloxy)quinazolin-4-amine (saracatinib), 4-[(2,4-dichloro-5-methoxyphenyl)amino]-6-methoxy-7-[3-(4-methylpiperazin-1-yl)propoxy]quinoline-3-carbonitrile (bosutinib), (4-Amino-5-(4-methylphenyl)-7-(t-butyl)pyrazolo[3,4-d]-pyrimidine), (4-chlorophenyl)-7-(t-butyl)pyrazolo[3,4-d]-pyrimidine) (PP1), 1-tert-butyl-3-(4-chlorophenyl)pyrazolo[3,4-d]pyrimidin-4-amine (PP2), 6-(2,6-dichlorophenyl)-2-{[3-(hydroxymethyl)phenyl]amino}-8-methyl-7H,8H-pyrido[2,3-d]pyrimidin-7-one (PD1663266), (E)-N-[4-[3-chloro-4-(pyridin-2-ylmethoxy)anilino]-3-cyano-7-ethoxyquinolin-6-yl]-4-(dimethylamino)but-2-enamide (neratinib), 3-(2-imidazo[1,2-b]pyridazin-3-ylethynyl)-4-methyl-N-[4-[(4-methylpiperazin-1-yl)methyl]-3-(trifluoromethyl)phenyl]benzamide (ponatinib), (E)-N-[4-(3-chloro-4-fluoroanilino)-3-cyano-7-ethoxyquinolin-6-yl]-4-(dimethylamino)but-2-enamide (pelitinib), N-benzyl-2-[5-[4-(2-morpholin-4-ylethoxy)phenyl]pyridin-2-yl]acetamide (Tirbanibulin), 4-methyl-3-[(2-methyl-6-pyridin-3-ylpyrazolo[3,4-d]pyrimidin-4-yl)amino]-N-[3-(trifluoromethyl)phenyl]benzamide (NVP-BHG712), (2S,3S)-2,3-dihydroxybutanedioic acid; 6-(4-methylpiperazin-1-yl)-N-(5-methyl-1H-pyrazol-3-yl)-2-[(E)-2-phenylethenyl]pyrimidin-4-amine (ENMD-2076), 4-[4-[(5-tert-butyl-2-quinolin-6-ylpyrazol-3-yl)carbamoylamino]-3-fluorophenoxy]-N-methylpyridine-2-carboxamide (Rebastinib), analogues thereof, or any combination thereof.

16. The method of claim 13, wherein the Src family kinase inhibitor is administered to the subject at a dose of less than about 150 mg per day and/or at an amount effective to provide a plasma drug level less than about 250 ng/ml.

17. The method of claim 13, further comprising administering an antimicrobial agent and/or EGFR inhibitor in combination with the Src family kinase inhibitor.

18. The method of claim 17, wherein the antimicrobial agent is an antibiotic agent, antiprotozoal agent, and/or antiparasitic agent.

19. The method of claim 17, wherein the antimicrobial agent and/or EGFR inhibitor is administered at a subtherapeutic dose with a subtherapeutic dose of the Src family kinase inhibitor, wherein the killing and/or degradation effect of the Src inhibitor on T. gondii is enhanced as compared to the effect of the Src inhibitor administered without the antimicrobial agent and/or EGFR inhibitor.

20. The method of claim 17, wherein the antimicrobial agent comprises at least one of at least one of amoxicillin, atovaquone, diaminopyrimidines, especially amodiaquine, amphotericin, proguanil, chloroquine, clindamycin, eflornithine, furazolidone, a fluoroquinolone, a third generation cephalosporin, hydroxychloroquine, mefloquine, melarsoprol, metronidazole, minocycline, nifursemizone, nitazoxanide, ornidazole, paromycin sulfate, pentamidine, pyrimethamine, quinapyramine, ronidazole, tinidazole, spriramycin, sulfadiazine, sulfamethoxazole, trimethoprim, analogues thereof, or combinations thereof.


SEQUENCE LISTING (4 sequences)

SEQ ID NO: 1 - DNA, 21 bp, Mus musculus
  source 1..21: mol_type = other DNA
gggagaacctggtgtgcaatt

SEQ ID NO: 2 - DNA, 21 bp, Mus musculus
  source 1..21: mol_type = other DNA
gtatagagcgtgcagataatt

SEQ ID NO: 3 - DNA, 20 bp, synthetic construct
  source 1..20: mol_type = other DNA
aacagagccccgaatgactg

SEQ ID NO: 4 - DNA, 20 bp, synthetic construct
  source 1..20: mol_type = other DNA
cgttttcggacaatgtggcg